(19) United States
(12) Patent Application Publication
(10) Pub. No.: US 2026/0284112 A1
(43) Pub. Date: Sep. 24, 2026

(54) ANTI-CLDN18.2 ANTIBODIES AND DIAGNOSTIC USES THEREOF

(71) Applicant: ANTENGENE BIOLOGICS LIMITED, Shanghai (CN)

(72) Inventors: Jishun CHEN, Shanghai (CN); Peng CHEN, Shanghai (CN); Bing HOU, Shanghai (CN); Bo SHAN, Shanghai (CN)

(21) Appl. No.: 19/490,942

(22) PCT Filed: Aug. 24, 2023
(86) PCT No.: PCT/CN2023/114671

§ 371 Date: Dec. 7, 2025

(30) Foreign Application Priority Data
Jun. 8, 2023 (WO) PCT/CN2023/099100

(51) Int. Cl. A61K 35/17; A61K 40/11; A61K 40/15; A61K 40/31; A61K 47/68; C07K 14/705; C07K 16/28; G01N 33/575

(52) U.S. Cl. CPC A61K 35/17; A61K 40/11; A61K 40/15; A61K 40/31; A61K 47/6849; C07K 14/7051; C07K 14/70514; C07K 14/70517; C07K 14/70521; C07K 16/28; G01N 33/5759; A61K 2239/13; A61K 2239/21; A61K 2239/22; C07K 2317/24; C07K 2317/33; C07K 2317/34; C07K 2317/52; C07K 2317/565; C07K 2317/622; G01N 2333/705

(57) ABSTRACT

The present disclosure provides anti-CLDN18.2 antibodies or antigen-binding fragments thereof, pharmaceutical com-position comprising the same and the uses thereof (e.g., diagnostic use).

### FIELD OF THE INVENTION

The present disclosure generally relates to novel anti-CLDN18.2 antibodies and diagnostic uses thereof.

### BACKGROUND

Claudins (CLDN) are a family of integral membrane proteins, which comprise a major structural protein of tight junctions in polarized cell types such as epithelial or endothelial cell sheets, and have been found to be a biological marker of various tumors.

CLDNs undergo endocytosis and the turnover time of some CLDNs is short relative to other membrane proteins (Van Raffle et al., J. Membrane Biol. 199, 29-38 (2004), PMID: 15366421). The expression of CLDNs is dysregulated in cancer cells and tight junction structures among tumor cells are disrupted in cancer cells. These properties allow antibodies to selectively bind claudin proteins in neoplastic but not in normal tissues. While antibodies specific to individual claudins are useful, it is also possible that polyreactive claudin antibodies or anti-pan claudin antibodies would be more likely to facilitate the delivery of payloads to a broader patient population due to higher aggregate antigen density that reduces the likelihood of escape of tumor cells with low levels of antigen expression of any individual claudin.

CLDN18.2, the isoform 2 of CLDN18, is physiologically confined to gastric mucosa tight junctions, the epitopes of which would be exposed on the cancer cell surface upon malignant transformation and is highly expressed in a significant proportion of cancers, e.g., gastric and pancreatic adenocarcinomas, which makes it a potential drug target for the treatment of cancers, e.g., gastric and pancreatic adenocarcinoma.

To enable screening and selection of patients with CLDN18.2 expression, there is a need for developing diagnostic anti-CLDN18.2 antibodies with high specificity and affinity.

### BRIEF SUMMARY OF THE INVENTION

The present disclosure provides, among others, novel anti-CLDN18.2 antibodies which specifically recognize CLDN18.2 without cross-reacting with CLDN18.1. The present disclosure further provides nucleotide sequences encoding such anti-CLDN18.2 antibodies, and use of such anti-CLDN18.2 antibodies, for example, for diagnostic purposes.

In one aspect, the present disclosure provides an antibody or antigen-binding fragment thereof comprising:
a) one, two or three heavy chain complementarity determining regions (HCDR1, HCDR2 and/or HCDR3) contained within a heavy chain variable (VH) region sequence as set forth in SEQ ID NO: 7; and/or
b) one, two or three light chain complementarity determining regions (LCDR1, LCDR2 and/or LCDR3) contained within a light chain variable (VL) region sequence as set forth in SEQ ID NO: 8.

In certain embodiments, the antibody or antigen-binding fragment thereof provided herein comprises at least one heavy or light chain complementarity determining region (CDR) comprising an amino acid sequence selected from the group consisting of SEQ ID NOs: 1, 2, 3, 4, 5 and 6.

In certain embodiments, the antibody or antigen-binding fragment thereof provided herein comprises one, two or three of HCDR1, HCDR2 and HCDR3 comprising an amino acid sequence selected from the group consisting of SEQ ID NOs: 1, 2 and 3.

In certain embodiments, the antibody or antigen-binding fragment thereof provided herein comprises one, two or three of LCDR1, LCDR2 and LCDR3 comprising an amino acid sequence selected from the group consisting of SEQ ID NOs: 4, 5 and 6.

In certain embodiments, the antibody or antigen-binding fragment thereof provided herein comprises:
a HCDR1 comprising an amino acid sequence as set forth in SEQ ID NO: 1;
a HCDR2 comprising an amino acid sequence as set forth in SEQ ID NO: 2; and
a HCDR3 comprising an amino acid sequence as set forth in SEQ ID NO: 3.

In certain embodiments, the antibody or antigen-binding fragment thereof provided herein comprises:
a LCDR1 comprising an amino acid sequence as set forth in SEQ ID NO: 4;
a LCDR2 comprising an amino acid sequence as set forth in SEQ ID NO: 5; and
a LCDR3 comprising an amino acid sequence as set forth in SEQ ID NO: 6.

In certain embodiments, the antibody or antigen-binding fragment thereof provided herein comprises:
a HCDR1 comprising an amino acid sequence as set forth in SEQ ID NO: 1, a HCDR2 comprising an amino acid sequence as set forth in SEQ ID NO: 2, a HCDR3 comprising an amino acid sequence as set forth in SEQ ID NO: 3, a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6.

In certain embodiments, the antibody or antigen-binding fragment thereof provided herein comprises a VH region having an amino acid sequence as set forth in SEQ ID NO: 7, or a homologous sequence thereof having at least 80% sequence identity to SEQ ID NO: 7.

In certain embodiments, the antibody or antigen-binding fragment provided herein comprises a VL region having an amino acid sequence as set forth in SEQ ID NO: 8, or a homologous sequence thereof having at least 80% sequence identity to SEQ ID NO: 8.

In certain embodiments, the antibody or antigen-binding fragment thereof provided herein comprises a VH region having an amino acid sequence as set forth in SEQ ID NO: 7 and a VL region having an amino acid sequence as set forth in SEQ ID NO: 8.

In certain embodiments, the antibody or antigen-binding fragment thereof provided herein further comprises one or more amino acid residue substitutions or modifications yet retains specific binding affinity to CLDN18.2.

In certain embodiments, the antibody or antigen-binding fragment thereof provided herein comprises one or more amino acid residue substitutions, wherein at least one of the substitutions or modifications is in one or more of the CDR sequences of the VH region or VL region.

In certain embodiments, the antibody or antigen-binding fragment provided herein comprises one or more amino acid residue substitutions, wherein at least one of the substitutions or modifications is in one or more of the non-CDR sequences of the VH region or VL region.

In certain embodiments, the antibody or antigen-binding fragment thereof provided herein further comprises one or more non-natural amino acid (NNAA) substitutions.

In certain embodiments, the antibody or antigen-binding fragment thereof provided herein comprises one or more non-natural amino acid (NNAA) substitutions, wherein the NNAA is capable of being conjugated.

In another aspect, the present disclosure provides an antibody or antigen-binding fragment thereof having one or more properties selected from the group consisting of:
(a) having substantially no cross-reactivity to human CLDN18.1;
(b) having substantially no cross-reactivity to human CLDN18.1 as measured by ELISA, FACS or IHC;
(c) having substantially no cross-reactivity to human CLDN18.1 peptide fragment within the amino acid sequence of STQDLYDNPVTSVFQYEGLWRSC (SEQ ID NO: 14) as measured by ELISA;
(d) being capable of specifically binding to an epitope within the amino acid sequence of TQDLYNNPVTAVFNYQGLWRSC (SEQ ID NO: 13) as measured by ELISA;
(e) being capable of specifically binding to an epitope within the amino acid sequence of human CLDN18.2 expressed in cells as measured by FACS assay; and
(f) being capable of specifically binding to human CLDN18.2 expressed in a cancer cell (e.g., a gastric cancer cell, a colon cancer cell, a hepatocellular carcinoma cell, an ovarian cancer cell, a cholangiocarcinoma cell, a pancreatic cancer cell, a breast cancer cell) as measured by IHC.

In certain embodiments, the antibody or antigen-binding fragment thereof provided herein competes for binding to CLDN18.2 with another antibody or antigen-binding fragment thereof that specifically binds to CLDN18.2.

In certain embodiments, the antibody or antigen-binding fragment thereof provided herein is a chimeric, a humanized or a human antibody or an antigen-binding fragment thereof.

In certain embodiments, the antibody or an antigen-binding fragment thereof provided herein is a monoclonal antibody, a bispecific antibody, a multi-specific antibody, a recombinant antibody, a chimeric antibody, a humanized antibody, a labeled antibody, a bivalent antibody, an anti-idiotypic antibody, a fusion protein, a dimerized or polymerized antibody, or a modified antibody (e.g., glycosylated antibody).

In certain embodiments, the antibody or antigen-binding fragment thereof provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a camelized single domain antibody, a nanobody, a domain antibody, or a bivalent domain antibody.

In certain embodiments, the antibody or antigen-binding fragment thereof provided herein further comprises an Fc region. In certain embodiments, the Fc region is an Fc region of human immunoglobulin (Ig). In certain embodiments, the Fc region is an Fc region of human IgG.

In certain embodiments, the antibody or antigen-binding fragment thereof provided herein comprises an Fc region, wherein the Fc region is derived from human IgG1, IgG2, IgG3 or IgG4, mouse IgG1 or rabbit IgG.

In certain embodiments, the antibody or antigen-binding fragment thereof provided herein comprises a light chain, wherein the light chain is a λ light chain or a κ light chain.

In certain embodiments, the antibody or antigen-binding fragment thereof comprises an Fc region, wherein the Fc region comprises an amino acid sequence of SEQ ID NO: 15, 16, 22, or a sequence having at least 80% sequence identity thereof, and/or the light chain comprises an amino acid sequence of SEQ ID NO: 19, 20, 21 or a sequence having at least 80% sequence identity thereof.

In certain embodiments, the antibody or antigen-binding fragment thereof provided herein is linked to one or more conjugate moieties.

In certain embodiments, the antibody or antigen-binding fragment thereof provided herein is linked to one or more conjugate moieties, wherein the conjugate moiety comprises a radioactive isotope, a lanthanide, a chemiluminescent label, a chromophoric moiety, colloidal gold particles, a detectable label, a fluorescent label, an enzyme-substrate label, a digoxigenin label, biotin/avidin, a hapten, a DNA molecule for detection, a particle label, a clearance-modifying agent, a chemotherapeutic agent, a toxin, a DNA-alkylator, a topoisomerase inhibitor, a tubulin-binder, a purification moiety or other anticancer drugs.

In certain embodiments, the antibody or antigen-binding fragment thereof provided herein is linked to one or more conjugate moieties, wherein the conjugate moiety comprises a biotin or a hapten.

In certain embodiments, the antibody or antigen-binding fragment thereof provided herein is linked to one or more conjugate moieties, wherein the conjugate moiety is covalently attached either directly or via a linker.

In another aspect, the present disclosure provides a chimeric antigen receptor comprising the antibody or antigen-binding fragment provided herein, a transmembrane region and an intracellular signal region. In certain embodiments, the transmembrane region of the chimeric antigen comprises a transmembrane region of CD3, CD4, CD8 or CD28. In certain embodiments, the intracellular signal region of the chimeric antigen receptor is selected from the group consisting of: an intracellular signal regions sequence of CD3, FcγRI, CD27, CD28, CD137, CD134, MyD88, CD40, CD278, TLRs, or a combination thereof. In certain embodiments, the antigen-binding fragment of the chimeric antigen receptor is a scFv.

In certain embodiments, the chimeric antigen receptor is grafted onto an allogeneic cell, an autologous cell or a xenogeneic cell. In certain embodiments, the chimeric antigen receptor is grafted onto an immune effector cell. In certain embodiments, the chimeric antigen receptor is grafted onto a T cell, a natural killer cell, a macrophage cell, or a tumor-infiltrating lymphocyte.

In another aspect, the present disclosure provides a pharmaceutical composition comprising the antibody or an antigen-binding fragment or the chimeric antigen receptor provided herein that specifically binds to CLDN18.2, and one or more pharmaceutically acceptable carriers.

In another aspect, the present disclosure provides an isolated polynucleotide encoding the antibody or an antigen-binding fragment thereof provided herein, and/or the chimeric antigen receptor.

In another aspect, the present disclosure provides a vector comprising the isolated polynucleotide encoding the antibody or an antigen-binding fragment thereof, and/or the chimeric antigen receptor provided herein.

In another aspect, the present disclosure provides a host expression system comprising the vector provided herein or having the polynucleotide integrated into genome thereof.

In certain embodiments, the host expression system is a microorganism, a yeast, or a mammalian cell. In certain embodiments, the microorganism is selected from the group consisting of E. coli and B. subtilis. In certain embodiments, the yeast is Saccharomyces. In certain embodiments, the mammalian cell is selected from the group consisting of COS, CHO-S, CHO-K1, HEK-293, and 3T3 cells.

In another aspect, the present disclosure provides a kit comprising the antibody or antigen-binding fragment thereof, and/or the chimeric antigen receptor, and/or the pharmaceutical composition provided herein. In certain embodiments, the kit further comprises a second agent. In certain embodiments, the second agent of the kit is a diagnostic agent. In certain embodiments, the kit further comprises a set of reagents for detecting a complex of the antibody or antigen-binding fragment thereof provided herein bound to CLDN18.2.

In certain embodiments, the set of reagents in the kit comprises a secondary antibody that binds to the antibody or antigen-binding fragment thereof, optionally the secondary antibody is detectably labeled. In certain embodiments, the set of reagents in the kit comprises a detectably labeled avidin or streptavidin.

In certain embodiments, the antibody or antigen binding fragment thereof provided herein is conjugated with an indirectly detectable moiety. In certain embodiments, the indirectly detectable moiety comprises biotin.

In another aspect, the present disclosure provides a method of expressing the antibody or antigen-binding fragment thereof or the chimeric antigen receptor provided herein, comprising culturing the host expression system under the condition at which the antibody or antigen-binding fragment or the chimeric antigen receptor is expressed.

In another aspect, the present disclosure provides a method of detecting presence or expression level of CLDN18.2 in a sample, comprising contacting the sample with the antibody or antigen-binding fragment thereof provided herein under a condition that allows specific binding of the antibody or antigen-binding fragment thereof to CLDN18.2, and determining presence or expression level of CLDN18.2 in the sample.

In another aspect, the present disclosure provides a method for diagnosing a CLDN18.2-associated disease, disorder or condition (e.g., cancer), screening for the onset or risk to the onset of a CLDN18.2-associated disease, disorder or condition (e.g., cancer), or assessing the development or prognosis of a CLDN18.2-associated disease, disorder or condition (e.g., cancer) in a subject, comprising:
a) contacting a sample obtained from the subject with the antibody or antigen-binding fragment thereof or the chimeric antigen receptor provided herein under a condition that allows specific binding of the antibody or antigen-binding fragment thereof or the chimeric antigen receptor to CLDN18.2; and
b) determining presence or expression level of CLDN18.2 in the sample;
   wherein the subject is diagnosed as having a CLDN18.2-associated disease, disorder or condition (e.g., cancer), or is at the onset or at a risk to the onset of a CLDN18.2-associated disease, disorder or condition (e.g., cancer), or develops or with an increased probability of developing a CLDN18.2-associated disease, disorder or condition (e.g., cancer), or has poor prognosis or at a risk to poor prognosis of a CLDN18.2-associated disease, disorder or condition (e.g., cancer) when the presence of CLDN18.2 is found or when the expression level of CLDN18.2 equals to or is higher than a threshold level.

In another aspect, the present disclosure provides a method for determining the eligibility of a subject having or at risk of having a CLDN18.2-associated disease, disorder or condition for treatment with a CLDN18.2-targeting agent, comprising:
a) contacting a sample obtained from the subject with the antibody or antigen-binding fragment thereof or the chimeric antigen receptor provided herein under a condition that allows specific binding of the antibody or antigen-binding fragment thereof or the chimeric antigen receptor to CLDN18.2; and
b) determining presence or expression level of CLDN18.2 in the sample;
   wherein the subject is determined as eligible for treatment with a CLDN18.2-targeting agent when the presence of CLDN18.2 is found or when the expression level of CLDN18.2 equals to or is higher than a threshold level, or
   wherein the subject is determined as not eligible for treatment with a CLDN18.2-targeting agent when the presence of CLDN18.2 is not found or when the expression level of CLDN18.2 is lower than a threshold level.

In another aspect, the present disclosure provides a method of predicting therapeutic effectiveness of a CLDN18.2-targeting agent in treating a CLDN18.2-associated disease, disorder or condition in a subject, comprising:
(a) contacting a sample obtained from the subject with the antibody or antigen-binding fragment thereof or the chimeric antigen receptor provided herein under a condition that allows specific binding of the antibody or antigen-binding fragment thereof or the chimeric antigen receptor to CLDN18.2;
(b) determining presence or expression level of CLDN18.2 in the sample; and
(c) predicting the therapeutic effectiveness of the CLDN18.2-targeting agent,
   wherein the CLDN18.2-targeting agent is predicted to be effective in treating the subject when the presence of CLDN18.2 is found or when the expression level of CLDN18.2 equals to or is higher than a threshold level, or
   wherein the CLDN18.2-targeting agent is predicted to be not effective in treating the subject when the presence of CLDN18.2 is not found or when the expression level of CLDN18.2 is lower than the threshold level.

In another aspect, the present disclosure provides a method of treating, preventing or alleviating a CLDN18.2-associated disease, disorder or condition in a subject having or at risk of having the CLDN18.2-associated disease, disorder or condition, comprising:
(a) selecting a subject that is suitable for the treatment, prevention or alleviation, comprising:
   (i). contacting a sample obtained from the subject with the antibody or antigen-binding fragment thereof or the chimeric antigen receptor provided herein under a condition that allows specific binding of the antibody or antigen-binding fragment thereof or the chimeric antigen receptor to CLDN18.2;
   (ii). determining the presence or expression level of CLDN18.2 in the sample; and
   (iii). selecting the subject as suitable for the treatment, prevention or alleviation of the CLDN18.2-associated disease, disorder or condition when the presence of CLDN18.2 is found or when the expression level of CLDN18.2 in the sample equals to or is higher than a threshold level;
(b) administering a therapeutically effective amount of CLDN18.2-targeting agent to the selected subject.

In another aspect, the present disclosure provides a method of treating, preventing or alleviating a CLDN18.2-associated disease, disorder or condition in a subject having or at risk of having the CLDN18.2-associated disease, disorder or condition, comprising:
(a) selecting a subject that is suitable for the treatment, prevention or alleviation, comprising:
   (i). contacting a sample obtained from the subject with the antibody or antigen-binding fragment thereof or the chimeric antigen receptor provided herein under a condition that allows specific binding of the antibody or antigen-binding fragment thereof or the chimeric antigen receptor to CLDN18.2;
   (ii). determining the presence or expression level of CLDN18.2 in the sample; and
   (iii). selecting the subject as not suitable for the treatment, prevention or alleviation of the CLDN18.2-associated disease, disorder or condition with a CLDN18.2-targeting agent when the presence of CLDN18.2 is not found or when the expression level of CLDN18.2 in the sample is lower than a threshold level;
(b) administering to the selected subject a Standard-of-Care therapeutic other than a CLDN18.2-targeting agent.

In certain embodiments, the sample is selected from the group consisting of a cell sample, a tissue sample, biopsy, a paraffin-embedded tissue, a body fluid (e.g., whole blood, blood serum, blood plasma, urine, mucus, saliva, peritoneal fluid, pleural fluid, chest fluid, synovial fluid, cerebrospinal fluid, thoracentesis fluid, abdominal fluid, and any combination thereof), colonic effluent, a surgical resection sample, an isolated blood cell, a cell isolated from blood, and any combination thereof. In certain embodiments, the sample is a fixed tissue sample, optionally a formalin-fixed paraffin-embedded (FFPE) tissue sample.

In certain embodiments, the CLDN18.2 is cell surface or membrane-bound CLDN18.2.

In certain embodiments, the presence or expression level of CLDN18.2 is determined by immunohistochemistry (IHC), Immunocytochemistry (ICC), immunofluorescence (IF), enzyme immunoassay (EIA), Enzyme-Linked Immunosorbent Assay (ELISA), immunochromatography, or immunoblotting.

In certain embodiments, the expression level of CLDN18.2 is quantified based on percentage of positively stained cells in the sample. In certain embodiments, the expression level of CLDN18.2 is quantified based on staining intensity for CLDN18.2 in the sample.

In certain embodiments, the CLDN18.2-associated disease, disorder or condition is cancer. In certain embodiments, the sample comprises a tumor sample. In certain embodiments, the tumor sample comprises a tumor tissue or a circulating tumor cell. In certain embodiments, the cancer is primary cancer or metastatic cancer.

In certain embodiments, the cancer is gastric cancer, colon cancer, hepatocellular carcinoma, ovarian cancer, cholangiocarcinoma, pancreatic cancer, breast cancer, lung cancer (e.g., non-small cell lung cancer or small cell lung cancer), bronchial cancer, bone cancer, liver and bile duct cancer, liver cancer, testicle cancer, kidney cancer, bladder cancer, head and neck cancer, spine cancer, brain cancer, cervix cancer, uterine cancer, endometrial cancer, colorectal cancer, rectal cancer, anal cancer, esophageal cancer, gastrointestinal cancer, skin cancer, prostate cancer, pituitary cancer, vagina cancer, thyroid cancer, glioblastoma, astrocytoma, melanoma, myelodysplastic syndrome, sarcoma, teratoma, or adenocarcinoma.

In certain embodiments, the cancer is gastric cancer, colon cancer, hepatocellular carcinoma, ovarian cancer, cholangiocarcinoma, pancreatic cancer, breast cancer, or lung cancer (e.g., non-small cell lung cancer or small cell lung cancer).

In certain embodiments, the CLDN18.2-targeting agent is capable of inducing cytotoxicity to CLDN18.2-expressing cells.

In certain embodiments, the CLDN18.2-targeting agent is a therapeutic anti-CLDN18.2 antibody or an CLDN18.2-binding molecule (e.g., anti-CLDN18.2 antibody conjugated to a cytotoxic agent, or a bispecific antibody), a CLDN18.2-targeting cell therapy (e.g., a CAR-T, TCR-T or CAR-NK cell expressing a CLDN18.2-binding CAR), a chemical compound targeting CLDN18.2, or a therapeutic nucleic acid targeting CLDN18.2.

In certain embodiments, the subject is receiving or has received anti-cancer therapy, or suffers from cancer recurrence. In certain embodiments, the subject is human. In certain embodiments, the CLDN18.2 is human CLDN18.2.

### BRIEF DESCRIPTION OF FIGURES

FIG. 1 shows the workflow of the generation and characterization of anti-CLDN18.2 antibodies.

FIG. 2 shows the binding of 43F11 antibody to human CLDN18.2 peptide fragment (996656-BSA) and human CLDN18.1 peptide fragment (996660-BSA).

FIG. 3 shows the representative IHC staining of CHO-K1.hCLDN18.1 cells and CHO-K1.hCLDN18.2 cells by 43F11 antibody.

FIG. 4 shows the representative IHC staining of gastric cancer tissue by 43F11 antibody.

FIG. 5 shows the representative IHC staining of different cancer tissues (e.g., colon cancer tissue, hepatocellular carcinoma tissue, ovarian cancer tissue, cholangiocarcinoma tissue, pancreatic cancer tissue, breast cancer tissue) by 43F11 antibody.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entireties.

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “antibody” as used herein includes any immunoglobulin, monoclonal antibody, polyclonal antibody, multivalent antibody, bivalent antibody, monovalent antibody, multi-specific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain comprises a variable region (VH) and a first, second, and third constant region (CH1, CH2, CH3, respectively); mammalian light chains are classified as λ or K, while each light chain comprises a variable region (VL) and a constant region. The antibody has a “Y” shape, with the stem of the Y comprising the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, and HCDR3). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain). The present disclosure includes all antibodies and derivatives of antibodies as described herein which for the purposes of the invention are encompassed by the term “antibody”. The term “antibody derivatives” refers to any modified form of an antibody, e.g., a conjugate of the antibody and another agent, an antibody fragment, or a fusion protein comprising the antibody or the antibody fragment.

As used herein, the term “antigen-binding fragment” refers to an antibody fragment formed from a portion of an antibody comprising one or more (e.g., 1, 2, 3, 4, 5, or 6) CDRs, or any other antibody fragment that binds to an antigen but does not comprise an intact native antibody structure. Examples of antigen-binding fragments include, without limitation, a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multi-specific antibody fragment, a camelized single domain antibody, a nanobody, a domain antibody, or a bivalent domain antibody. An antigen-binding fragment is capable of binding to the same antigen or epitope to which the parent antibody binds. In certain embodiments, an antigen-binding fragment may comprise one or more CDRs from a particular antibody.

“Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

“Fab′” refers to a Fab fragment that includes a portion of the hinge region. Fab′ can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab′)₂” refers to a dimer of Fab′ that comprises two light chains and part of two heavy chains.

“Fc” with regard to an antibody (e.g., of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a linker (e.g., a peptide sequence) (Huston J S et al. Proc Natl Acad Sci USA, 85: 5879 (1988)).

A “scFv dimer” refers to a single chain comprising two heavy chain variable regions and two light chain variable regions with a linker. In certain embodiments, an “scFv dimer” is a bivalent diabody or bivalent scFv (BsFv) comprising VH-VL (linked by a peptide linker) dimerized with another VH-VL moiety such that VH's of one moiety coordinate with the VL's of the other moiety and form two binding sites which can target the same antigens (or epitopes) or different antigens (or epitopes). In other embodiments, a “scFv dimer” is a bispecific diabody comprising VH1-VL2 (linked by a peptide linker) associated with VL1-VH2 (also linked by a peptide linker) such that VH1 and VLI coordinate and VH2 and VL2 coordinate and each coordinated pair has a different antigen specificity.

“Single-chain Fv-Fc antibody” or “scFv-Fc” refers to an engineered antibody consisting of a scFv connected to the Fc region of an antibody.

“Camelized single domain antibody,” “heavy chain antibody,” “nanobody” or “HCAb” refers to an antibody that contains two VH domains and no light chains (Riechmann L. and Muyldermans S., J Immunol Methods. December 10; 231 (1-2): 25-38 (1999); Muyldermans S., J Biotechnol. June; 74 (4): 277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363 (6428): 446-8 (1993); Nguyen V K. et al. “Heavy-chain antibodies in Camelidae; a case of evolutionary innovation,” Immunogenetics. April; 54 (1): 39-47 (2002); Nguyen V K. et al. Immunology. May; 109 (1): 93-101 (2003)). The variable domain of a heavy chain antibody (VHH domain) represents the smallest known antigen-binding unit generated by adaptive immune responses (Koch-Nolte F. et al., FASEB J. November; 21 (13): 3490-8. Epub 2007 Jun. 15 (2007)).

A “diabody” or “dAb” includes small antibody fragments with two antigen-binding sites, wherein the fragments comprise a VH domain connected to a VL domain in the same polypeptide chain (VH-VL or VL-VH) (see, e.g., Holliger P. et al., Proc Natl Acad Sci USA. July 15; 90 (14): 6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain embodiments, two or more VH domains are covalently joined with a peptide linker to form a bivalent or multivalent domain antibody. The two VH domains of a bivalent domain antibody may target the same or different antigens.

A “(dsFv)₂” refers to disulfide-stabilized Fv fragments comprising three peptide chains: two VH moieties linked by a peptide linker and bound by disulfide bridges to two VL moieties.

The term “humanized” as used herein means that the antibody or antigen-binding fragment comprises CDRs derived from non-human animals, FR regions derived from human, and when applicable, constant regions derived from human.

The term “chimeric” as used herein refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

“Anti-CLDN18.2 antibody” or “an antibody against CLDN18.2” as used herein refers to an antibody that is capable of specific binding to CLDN18.2 (e.g., human or non-human CLDN18.2) with a sufficient affinity, for example, to provide for diagnostic and/or therapeutic use.

The term “affinity” as used herein refers to the strength of non-covalent interaction between an immunoglobulin molecule (i.e., antibody) or fragment thereof and an antigen.

The term “specific binding” or “specifically binds” or “binding specificity” as used herein refers to a non-random binding reaction between two molecules, such as for example between an antibody and an antigen. An antibody or antigen-binding fragment thereof that “specifically binds” or “specific binding” to a target (e.g., an epitope) is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. For example, an antibody that specifically binds to a CLDN18.2 epitope is an antibody that binds this CLDN18.2 epitope with greater affinity, avidity, more readily, and/or with greater duration than it binds to other CLDN18.2 epitopes or non-CLDN18.2 epitopes. It is also understood by reading this definition that, for example, an antibody (or moiety or epitope) that specifically binds to a first target may or may not specifically bind to a second target. As such, “specific binding” or “specifically bind” does not necessarily require (although it can include) exclusive binding. Generally, but not necessarily, reference to binding means specific binding.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23 (21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among amino acid residues with neutral hydrophilic side chains (e.g., Cys, Ser, Thr, Asn and Gln), among amino acid residues with acidic side chains (e.g., Asp, Glu), among amino acid residues with basic side chains (e.g., His, Lys, and Arg), or among amino acid residues with aromatic side chains (e.g., Trp, Tyr, and Phe). As known in the art, conservative substitution usually does not cause significant change in the protein conformational structure, and therefore could retain the biological activity of a protein.

The term “homologous” as used herein refers to a nucleic acid sequence (or its complementary strand) or an amino acid sequence that has sequence identity of at least 60% (e.g., at least 65%, 70%, 75%, 80%, 85%, 88%, 90%, 91%, 92%, 93%, 94%, 95%, 96%, 97%, 98%, or 99%) to another sequence when optimally aligned.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated”, but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% as determined by electrophoretic methods (such as SDS-PAGE, isoelectric focusing, capillary electrophoresis), or chromatographic methods (such as ion exchange chromatography or reverse phase HPLC).

The term “subject” includes human and non-human animals. Non-human animals include all vertebrates, e.g., mammals and non-mammals, such as non-human primates, rodent (e.g., mouse, rat and guinea pigs), cat, rabbit, sheep, dog, cow, chickens, amphibians, and reptiles. In more preferred embodiments, the subject is a human. Except when noted, the terms “patient”, “subject” and “individual” are used herein interchangeably.

“Effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and antibody dependent cell mediated phagocytosis (ADCP), where nonspecific cytotoxic cells that express FcγRs recognize bound antibody on a target cell and subsequently cause phagocytosis of the target cell. Effector functions include both those that operate after the binding of an antigen and those that operate independent of antigen binding.

“Treating” or “treatment” or “therapy” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

By “being at risk” is meant a subject, e.g., a patient, that is identified as having a higher than normal chance of developing a disease, in particular cancer, compared to the general population. In addition, a subject who has had, or who currently has, a disease, in particular cancer is a subject who has an increased risk for developing a disease, as such a subject may continue to develop a disease. Subjects who currently have, or who have had, a cancer also have an increased risk for cancer metastases. In the context of the present invention, terms such as “protect”, “prevent”, “prophylactic” relate to the prevention or treatment or both of the occurrence and/or the propagation of a disease in a subject and, in particular, to minimizing the chance that a subject will develop a disease or to delaying the development of a disease. For example, a person at risk of a tumor, as described above, would be a candidate for therapy to prevent a tumor. Immunotherapy may be performed using any of a variety of techniques, in which agents function to remove antigen-expressing cells from a patient.

“Standard-of-Care” therapeutics described herein comprise the administration of a standard-of-care therapeutic to a patient. As used herein, a “standard-of-care therapeutic” is a treatment process, including a drug or combination of drugs, radiation therapy (RT), surgery or other medical intervention that is recognized by medical practitioners as appropriate, accepted, and/or widely used for a certain type of patient, disease or clinical circumstance. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers (see NCCN GUIDELINES®, 2013).

The terms “effective” and “effectiveness” with regard to a treatment include both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

A “therapeutically effective amount” or “therapeutically effective dosage” of a drug or therapeutic agent, such as an antibody of the present disclosure, is any amount of the drug that, when used alone or in combination with another therapeutic agent, protects a subject against the onset of a disease or condition, or promotes disease/condition regression evidenced by a decrease in severity of disease/condition symptoms, an increase in frequency and duration of disease/condition symptom-free periods, or a prevention of impairment or disability due to the disease/condition affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays. A therapeutically effective amount of a drug includes a “prophylactically effective amount,” which is any amount of the drug that, when administered alone or in combination with an anti-neoplastic agent to a subject at risk of developing a CLDN18.2-associated disease or condition, such as a cancer (e.g., a subject having a pre-malignant condition) or of suffering a disease/condition recurrence, inhibits the development or recurrence of the disease/condition. In preferred embodiments, the prophylactically effective amount prevents the development or recurrence of the CLDN18.2-associated disease or condition (e.g., cancer) entirely. “Inhibiting” the development or recurrence of a disease/condition (e.g., cancer) means either lessening the likelihood of the disease/condition's development or recurrence, or preventing the development or recurrence of the disease/condition entirely.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or antigen-binding fragment thereof, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

The phrase “host cell” as used herein refers to a cell into which an exogenous polynucleotide and/or a vector can be or has been introduced.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis,” “diagnose,” or “diagnosing” refers to the identification of a pathological state, disease or condition, such as identification of a CLDN18.2 related disease or refer to identification of a subject with a CLDN18.2 related disease who may benefit from a particular treatment regimen. In some embodiments, diagnosis contains the identification of abnormal amount or activity of CLDN18.2. In some embodiments, diagnosis refers to the identification of a cancer in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from stomach, heart, liver, spleen, lung, kidney, skin or blood vessels of the subject.

A “CLDN18.2-associated” disease, disorder or condition as used herein refers to any disease or condition caused by, exacerbated by, or otherwise linked to increased or decreased expression or activities of CLDN18.2. In some embodiments, the CLDN18.2 related disease, disorder or condition is, for example, cancer.

The term “cancer” as used herein refers to any medical condition characterized by malignant cell growth or neoplasm, abnormal proliferation, infiltration or metastasis, and includes both solid tumors and non-solid cancers (e.g., hematologic malignancies) such as leukemia.

As used herein, the term “solid tumor” refers to a solid mass of neoplastic and/or malignant cells.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “metastasis” or “metastatic cancer” as used herein means the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e., a secondary tumor or metastatic tumor, at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor and the regional lymph node system. The cells of a secondary or metastatic tumor are like those in the original tumor. This means, for example, that, if gastric cancer metastasizes to the liver, the secondary tumor is made up of abnormal gastric cells, not of abnormal liver cells. The tumor in the liver is then called metastatic gastric cancer, not liver cancer.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g., within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g., about 1 year means 11-13 months; about 6 months means 6 months plus or minus 1 week; about 1 week means 6-8 days; etc.), or within 10 percent of the indicated value, whichever is greater.

### Anti-CLDN18.2 Antibodies

The present disclosure provides anti-CLDN18.2 antibodies and antigen-binding fragments thereof.

“CLDN18” as used herein refers to Claudin18 and includes any variants thereof, including CLDN18.1 and CLDN18.2, conformations, isoforms and species homologs of CLDN18 which are naturally expressed by cells or are expressed by cells transfected with the CLDN18 gene. In certain embodiments, the CLDN18 is human CLDN18. “CLDN18” as used herein may be from other animal species, such as from human, mouse, and cynomolgus, among others. The terms “CLDN18”, “CLDN-18”, “CLDN 18”, “Claudin18”, “Claudin-18”, or “Claudin 18” may be used interchangeably in the present disclosure.

CLDN18 has two different splice variants (CLDN18.1 and CLDN18.2), which are present in both mouse and human. The splice variants CLDN18.1 and CLDN18.2 differ in the first 21 amino acids at the N-terminus that comprises the first TM and the loop1, whereas the protein sequences in the C-terminus are identical. Although these two isoforms share 92% identity in amino acid sequence, their expression patterns are mutually exclusive with CLDN18.1 being predominantly expressed in normal lung and CLDN18.2 in normal gastric tissue (see Niimi T, et al., Molecular and cellular biology, 2001, 21 (21): 7380-7390.).

“CLDN18.2” is a splice variant of CLDN18, and includes post-translationally modified variants, isoforms and species homologs of CLDN18.2 which are naturally expressed by cells or are expressed on cells transfected with the CLDN18.2 gene. The terms “CLDN18.2”, “CLDN-18.2”, “CLDN 18.2”, “Claudin18.2”, “Claudin-18.2”, or “Claudin 18.2” may be used interchangeably in the present disclosure. Exemplary sequence of human CLDN18.2 amino acid sequence (Accession: NP_001002026.1) is as shown in SEQ ID NO: 9 below.

(SEQ ID NO: 9)
MAVTACQGLGFVVSLIGIAGIIAATCMDQWSTQDLYNNPVTAVFNYQGLW
RSCVRESSGFTECRGYFTLLGLPAMLQAVRALMIVGIVLGAIGLLVSIFA
LKCIRIGSMEDSAKANMTLTSGIMFIVSGLCAIAGVSVFANMLVTNFWMS
TANMYTGMGGMVQTVQTRYTFGAALFVGWVAGGLTLIGGVMMCIACRGLA
PEETNYKAVSYHASGHSVAYKPGGFKASTGFGSNTKNKKIYDGGARTEDE
VQSYPSKHDYV

“CLDN18.1” is a splice variant of CLDN18, and includes post-translationally modified variants, isoforms and species homologs of CLDN18.1 which are naturally expressed by cells or are expressed on cells transfected with the CLDN18.1 gene. The terms “CLDN18.1”, “CLDN-18.1”, “CLDN 18.1”, “Claudin18.1”, “Claudin-18.1”, or “Claudin 18.1” may be used interchangeably in the present disclosure. Exemplary sequence of human CLDN18.1 amino acid sequence (Accession: NP_057453.1) is as shown in SEQ ID NO: 23 below.

(SEQ ID NO: 23)
MSTTTCQVVAFLLSILGLAGCIAATGMDMWSTQDLYDNPVTSVFQYEGLW
RSCVRQSSGFTECRPYFTILGLPAMLQAVRALMIVGIVLGAIGLLVSIFA
LKCIRIGSMEDSAKANMTLTSGIMFIVSGLCAIAGVSVFANMLVTNFWMS
TANMYTGMGGMVQTVQTRYTFGAALFVGWVAGGLTLIGGVMMCIACRGLA
PEETNYKAVSYHASGHSVAYKPGGFKASTGFGSNTKNKKIYDGGARTEDE
VQSYPSKHDYV

In normal tissue, expression of CLDN18.2 is restricted to the basal membrane of mucosal cells and is not accessible to therapeutic antibodies. In pathologic conditions (such as tumor cells) the polarity of the gastric mucosa cells is perturbed and CLDN18.2 is exposed on the cell surface. CLDN18.2 protein is a pan-cancer target expressed in primary lesions and metastases of several human cancer types, including stomach, esophageal, pancreatic and lung tumors as well as human cancer cell lines (see Sahin Ugur et al., Clin Cancer Res 2008; 14 (23); Matsuda Y et al., Cancer Science, 2007, 98 (7): 1014-1019). Aberrant ectopic expression of CLDN18.2 has also been reported in pancreatic, ovarian, biliary and lung adenocarcinomas in multiple studies (see, for example, Karanjawala Z E et al., Am J Surg Pathol. 2008 February; 32 (2): 188-96; Micke P et al., Int J Cancer. 2014 Nov. 1; 135 (9): 2206-14; Keira Y et al., Virchows Arch. 2015 March; 466 (3): 265-77; Coati et al., Br J Cancer. 2019 July; 121 (3): 257-263; Dottermusch et al., Virchows Arch. 2019 November; 475 (5): 563-571; Rohde et al., Jpn J Clin Oncol. 2019 Sep. 1; 49 (9): 870-876; Woll et al., Int J Cancer. 2014 Feb. 1; 134 (3): 731-9; Espinoza et al., Histopathology. 2019 March; 74 (4): 597-607; Shinozaki et al., Virchows Arch. 2011 July; 459 (1): 73-80).

The present disclosure in one aspect provides monoclonal anti-CLDN18.2 antibodies and antigen-binding fragments thereof. The monoclonal anti-CLDN18.2 antibodies and antigen-binding fragments provided herein are capable of specifically binding to CLDN18.2 (e.g., human CLDN18.2), a fragment of CLDN18.2, or a fusion polypeptide comprising the fragment of CLDN18.2. In certain embodiments, the fragment of CLDN18.2 comprises the first extracellular loop of human CLDN18.2, or a sequence within the first extracellular loop of human CLDN18.2. In certain embodiments, the fragment of human CLDN18.2 comprises the amino acid sequence set forth in SEQ ID NO: 10, 11, 12 or 13. In certain embodiments, the fusion polypeptide comprises additional amino acid residues attached to the N terminal and/or C terminal of the fragment of CLDN18.2. In certain embodiments, the fragment of CLDN18.2 contained in the fusion polypeptide forms a loop.

The anti-CLDN18.2 antibodies and antigen-binding fragments provided herein are capable of specifically binding to CLDN18.2-expressing cells. In certain embodiments, the CLDN18.2-expressing cells are pre-treated cells. The term “pre-treated” as used herein with respect to cells means that the cells have been treated such that its surface proteins such as CLDN18.2 are denatured or otherwise are no longer in its native conformation. For example, the CLDN18.2-expressing cells can be pre-treated by one or more chemical substances, for example formalin, paraffin or acetone, or by physical intervention such as freezing or heating. In certain embodiments, the CLDN18.2-expressing cells are formalin-fixed paraffin-embedded (FFPE) cells.

Binding of the anti-CLDN18.2 antibodies and antigen-binding fragments provided herein to the antigen can be represented by “half maximal inhibitory concentration” (IC₅₀) value, which refers to the concentration of an antibody that is required for 50% inhibition to an antigen (e.g., human CLDN18.2). The IC₅₀ value can be measured by methods known in the art, for example, sandwich assay such as ELISA, Western Blot, and other binding assays. The IC₅₀ can be measured in a proper binding assay where serial dilutions of the antibody are tested for binding to the antigen, and the concentration at which 50% of inhibition is determined. In certain embodiments, the anti-CLDN18.2 antibody or the antigen-binding fragment thereof provided herein specifically binds to human CLDN18.2, a fragment of human CLDN18.2, a fusion polypeptide comprising the fragment of human CLDN18.2, human CLDN18.2-expressing cells or pre-treated human CLDN18.2-expressing cells. In certain embodiments, the anti-CLDN18.2 antibody or the antigen-binding fragment thereof provided herein specifically binds to a fusion polypeptide comprising the first extracellular loop of human CLDN18.2 at an IC₅₀ of no more than 1 nM (e.g., no more than 0.9 nM, no more than 0.8 nM, no more than 0.7 nM, no more than 0.6 nM, no more than 0.5 nM, no more than 0.4 nM, no more than 0.3 nM, no more than 0.2 nM, no more than 0.1 nM) as measured by ELISA.

In certain embodiments, the anti-CLDN18.2 antibodies and the antigen-binding fragments thereof provided herein have substantially no cross-reactivity to human CLDN18.1. An anti-CLDN18.2 antibody that “substantially does not cross-react with” or “has substantially no cross-reactivity to” human CLDN18.1 is an antibody that substantially do not produce undesired results such as false positives in a detection assay (e.g., IHC assay, ELISA assay, FACS assay) for human CLDN18.2. In certain embodiments, the binding to human CLDN18.1 is substantially not detectable in the detection assay (e.g., IHC assay, or flow cytometry assay). The level of binding can be determined as the level of antigen-antibody complex that is formed at a given concentration of the antibody and a given concentration of the antigen.

In certain embodiments, the anti-CLDN18.2 antibodies and the antigen-binding fragments thereof provided herein have substantially no cross-reactivity to human CLDN18.1 peptide fragment within the amino acid sequence of STQDLYDNPVTSVFQYEGLWRSC (SEQ ID NO: 14), e.g., as measured by ELISA assay. In certain embodiments, the anti-CLDN18.2 antibodies and the antigen-binding fragments thereof provided herein are capable of specifically binding to an epitope within the amino acid sequence of TQDLYNNPVTAVFNYQGLWRSC (SEQ ID NO: 13), e.g., as measured by ELISA assay.

In certain embodiments, no more than 5%, 4%, 3%, 2%, 1%, 0.8%, 0.5%, 0.3% or 0.1% of the human CLDN18.1-expressing cells are detected positive in an IHC assay by the anti-CLDN18.2 antibodies and the antigen-binding fragments thereof provided herein. In certain embodiments, the anti-CLDN18.2 antibodies and the antigen-binding fragments thereof provided herein do not show detectable binding to human CLDN18.1 in an IHC assay. In certain embodiments, the anti-CLDN18.2 antibodies and the antigen-binding fragments thereof provided herein do not show detectable binding to human CLDN18.1 in a non-cancerous human lung tissue sample in an IHC assay.

In certain embodiments, the anti-CLDN18.2 antibodies and the antigen-binding fragments thereof provided herein are capable of specifically binding to an epitope within the amino acid sequence of human CLDN18.2 expressed in cells as measured by FACS assay.

In certain embodiments, the anti-CLDN18.2 antibodies and the antigen-binding fragments thereof provided herein are capable of specifically binding to human CLDN18.2 expressed in a cancer cell (e.g., a gastric cancer cell, a colon cancer cell, a hepatocellular carcinoma cell, an ovarian cancer cell, a cholangiocarcinoma cell, a pancreatic cancer cell, a breast cancer cell) as measured by IHC assay. The IHC assay can be conducted by the protocol as outlined in Example 2 of the present disclosure.

### Illustrative Anti-CLDN18.2 Antibody

In one aspect, the present disclosure provides an antibody or antigen-binding fragment thereof which specifically binds to CLDN18.2, comprising:
(a) one, two or three heavy chain complementarity determining regions (HCDR1, HCDR2 and/or HCDR3) contained within a heavy chain variable (VH) region sequence as set forth in SEQ ID NO: 7; and/or
(b) one, two or three light chain complementarity determining regions (LCDR1, LCDR2 and/or LCDR3) contained within a light chain variable (VL) region sequence as set forth in SEQ ID NO: 8.

A person skilled in the art can define or identify CDR boundaries of a VH or VL region by well-known methods in the art as long as the amino acid sequence of the VH or VL region is known. For example, CDR boundaries for an antibody or antigen-binding fragment thereof may be defined or identified by the conventions of Kabat, IMGT, Chothia, or Al-Lazikani (Al-Lazikani, B., Chothia, C., Lesk, A. M., J. Mol. Biol., 273 (4), 927 (1997); Chothia, C. et al., J Mol Biol. December 5; 186 (3): 651-63 (1985); Chothia, C. and Lesk, A. M., J. Mol. Biol., 196,901 (1987); Chothia, C. et al., Nature. December 21-28; 342 (6252): 877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27:55-77 (2003); Marie-Paule Lefranc et al., Immunome Research, 1 (3), (2005); Marie-Paule Lefranc, Molecular Biology of B cells (second edition), chapter 26, 481-514, (2015)). In some embodiments, the CDR boundaries of the antibodies or antigen-binding fragments thereof provided herein are identified by the convention of Kabat. In some embodiments, the CDR boundaries of the antibodies or antigen-binding fragments thereof provided herein are identified by the convention of IMGT. In some embodiments, the CDR boundaries of the antibodies or antigen-binding fragments thereof provided herein are identified by the convention of Chothia. In some embodiments, the CDR boundaries of the antibodies or antigen-binding fragments thereof provided herein are identified by the convention of Al-Lazikani.

In certain embodiments, the antibody or antigen-binding fragment provided herein comprises at least one heavy or light chain complementarity determining region (CDR) comprising an amino acid sequence selected from the group consisting of SEQ ID NOs: 1, 2, 3, 4, 5 and 6.

In certain embodiments, the antibody or antigen-binding fragment thereof provided herein comprises one, two or three of HCDR1, HCDR2 and HCDR3 comprising an amino acid sequence selected from the group consisting of SEQ ID NOs: 1, 2 and 3.

In certain embodiments, the antibody or antigen-binding fragment thereof provided herein comprises one, two or three of LCDR1, LCDR2 and LCDR3 comprising an amino acid sequence selected from the group consisting of SEQ ID NOs: 4, 5 and 6.

In certain embodiments, the antibody or antigen-binding fragment thereof provided herein comprises:
a HCDR1 comprising an amino acid sequence as set forth in SEQ ID NO: 1;
a HCDR2 comprising an amino acid sequence as set forth in SEQ ID NO: 2; and
a HCDR3 comprising an amino acid sequence as set forth in SEQ ID NO: 3.

In certain embodiments, the antibody or antigen-binding fragment thereof provided herein comprises:
a LCDR1 comprising an amino acid sequence as set forth in SEQ ID NO: 4;
a LCDR2 comprising an amino acid sequence as set forth in SEQ ID NO: 5; and
a LCDR3 comprising an amino acid sequence as set forth in SEQ ID NO: 6.

In certain embodiments, the antibody or antigen-binding fragment thereof provided herein comprises:
a HCDR1 comprising an amino acid sequence as set forth in SEQ ID NO: 1,
a HCDR2 comprising an amino acid sequence as set forth in SEQ ID NO: 2,
a HCDR3 comprising an amino acid sequence as set forth in SEQ ID NO: 3,
a LCDR1 comprising an amino acid sequence as set forth in SEQ ID NO: 4,
a LCDR2 comprising an amino acid sequence as set forth in SEQ ID NO: 5,
and a LCDR3 comprising an amino acid sequence as set forth in SEQ ID NO: 6.

In certain embodiments, the antibody or antigen-binding fragment thereof provided herein comprises a VH region having an amino acid sequence as set forth in SEQ ID NO: 7, or a homologous sequence thereof having at least 80% sequence identity to SEQ ID NO: 7.

In certain embodiments, the antibody or antigen-binding fragment provided herein comprises a VL region having an amino acid sequence as set forth in SEQ ID NO: 8, or a homologous sequence thereof having at least 80% sequence identity to SEQ ID NO: 8.

In certain embodiments, the antibody or antigen-binding fragment thereof provided herein comprises a VH region having an amino acid sequence as set forth in SEQ ID NO: 7 and a VL region having an amino acid sequence as set forth in SEQ ID NO: 8.

CDRs are known to be responsible for antigen binding. However, it has been found that not all of the 6 CDRs are indispensable or unchangeable. In other words, it is possible to replace or change or modify one or more CDRs provided above (e.g., corresponding to any one of SEQ ID NOs: 1-6), yet substantially retain the specific binding affinity to CLDN18.2. Antibodies having such modified or variant CDRs are also encompassed in the present disclosure.

Antibody “43F11” as used herein refers to a mouse monoclonal antibody having a heavy chain variable region having the sequence as set forth in SEQ ID NO: 7, and a light chain variable region having the sequence as set forth in SEQ ID NO: 8.

The SEQ ID NOs and amino acid sequences of the HCDRs and LCDRs of antibody 43F11 are shown in Table 1 below. Unless otherwise indicated, the CDR boundaries as described in Table 1 below were defined or identified by the convention of Kabat. The SEQ ID NOs and amino acid sequences of the heavy chain variable region (denoted as “VH”) and light chain variable region (denoted as “VL”) of antibody 43F11 are shown in Table 2 below.

TABLE 1
SEQ ID NOs and amino acid sequences of HCDRs and LCDRs of
antibody 43F11

| | | CDR1 | CDR2 | CDR3 |
|---|---|---|---|---|
| Antibody | Region | (SEQ ID NO) | (SEQ ID NO) | (SEQ ID NO) |
| 43F11 | HCDR | SYGMH | FISSGSRTIYYADT | STGWDDWFVY |
| | | (SEQ ID NO: 1) | VKG | (SEQ ID NO: 3) |
| | | | (SEQ ID NO: 2) | |
| | LCDR | RSSKSLLHTNG | RMSNLAS | MQHLEYPLT |
| | | NTYLY | (SEQ ID NO: 5) | (SEQ ID NO: 6) |
| | | (SEQ ID NO: 4) | | |

TABLE 2
SEQ ID NOs and amino acid sequences of VH and VL regions of
antibody 43F11

| Antibody | VH | VL |
|---|---|---|
| 43F11 | SEQ ID NO: 7 | SEQ ID NO: 8 |
| | DVQLVESGGGLVQPGGSRKLS | DIVMTQSAPSVPVTPGESVSIS |
| | CAASGFTFSSYGMHWVRQAPE | CRSSKSLLHTNGNTYLYWFLQ |
| | KGLEWVAFISSGSRTIYYADTV | RPGQSPQVLIYRMSNLASGVP |
| | KGRFTISRDNPKNTLFLQMTSL | DRFSGSGSGTAFTLRISRVEAD |
| | RSEDTAMYYCAGSTGWDDWF | DVGVYYCMQHLEYPLTFGAG |
| | VYWGQGTLVTVSA | TKLELK |

In certain embodiments, the antibodies and antigen-binding fragments thereof provided herein comprise suitable framework region (FR) sequences, as long as the antibodies and antigen-binding fragments thereof can specifically bind to CLDN18.2 (e.g., human CLDN18.2). The CDR sequences provided in Table 1 are obtained from mouse antibodies, but they can be grafted to any suitable FR sequences of any suitable species such as mouse, human, rat, rabbit, among others, using suitable methods known in the art such as recombinant techniques. FR sequences can be readily identified by a skilled person in the art based on the CDR sequences in Table 1 above and variable region sequences in Table 2 above, as it is well-known in the art that a CDR region is flanked by two FR regions in the variable region.

In certain embodiments, the anti-CLDN18.2 antibody or an antigen-binding fragment thereof provided herein further comprises an immunoglobulin constant region. The constant region optionally comprises a heavy chain constant region of IgG, and/or a light chain constant region. The heavy chain constant region comprises CH1, hinge, and/or CH2-CH3 regions. In certain embodiments, the heavy chain constant region comprises an Fc region. In certain embodiments, the Fc region is an Fc region of human immunoglobulin (Ig). In certain embodiments, the Fc region is an Fc region of human IgG. In certain embodiments, the Fc region is derived from human IgG1, IgG2, IgG3 or IgG4, mouse IgG1 or rabbit IgG. In certain embodiments, the light chain constant region comprises Ck or Ch.

In certain embodiments, the anti-CLDN18.2 antibodies and the fragments thereof provided herein further comprise a constant region of mouse IgG1, IgG2, IgG3, or IgG4. In certain embodiments, the anti-CLDN18.2 antibodies and antigen-binding fragments thereof provided herein comprises a constant region of mouse IgG1 isotype. In certain embodiments, the anti-CLDN18.2 antibodies and the fragments thereof provided herein further comprise a Fc region comprising an amino acid sequence of SEQ ID NO: 15, 16, 22, or a sequence having at least 80% (e.g., at least 85%, 90%, 95%, 96%, 97%, 98%, or 99%) sequence identity thereof.

In certain embodiments, the anti-CLDN18.2 antibodies and the fragments thereof provided herein further comprise a light chain comprising an amino acid sequence of SEQ ID NO: 19, 20, 21, or a sequence having at least 80% (e.g., at least 85%, 90%, 95%, 96%, 97%, 98%, or 99%) sequence identity thereof.

The amino acid sequences of SEQ ID NOs: 15, 16, 22, 19, 20 and 21 are shown in Table 9 below.

TABLE 9
Amino acid sequences of Fc regions and light chain constant regions

| SEQ ID | | |
|---|---|---|
| NO: | Description | Amino Acid Sequence |
| 15 | mouse IgG1 Fc | VPEVSSVFIFPPKPKDVLTITLTPKVTCV |
| | | VVDISKDDPEVQFSWFVDDVEVHTAQT |
| | | QPREEQFNSTFRSVSELPIMHQDWLNGK |
| | | EFKCRVNSAAFPAPIEKTISKTKGRPKAP |
| | | QVYTIPPPKEQMAKDKVSLTCMITDFFP |
| | | EDITVEWQWNGQPAENYKNTQPIMDTD |
| | | GSYFVYSKLNVQKSNWEAGNTFTCSVL |
| | | HEGLHNHHTEKSLSHSPGK |
| 16 | Rabbit IgG Fc | VPELLGGPSVFIFPPKPKDTLMISRTPEV |
| | | TCVVVDVSQDDPEVQFTWYINNEQVRT |
| | | ARPPLREQQFNSTIRVVSTLPITHQDWLR |
| | | GKEFKCKVHNKALPAPIEKTISKARGQP |
| | | LEPKVYTMGPPREELSSRSVSLTCMING |
| | | FYPSDISVEWEKNGKAEDNYKTTPAVL |
| | | DSDGSYFLYNKLSVPTSEWQRGDVFTC |
| | | SVMHEALHNHYTQKSISRSPGK |
| 22 | human IgG1 Fc | TCPPCPAPELLGGPSVFLFPPKPKDTLMI |
| | | SRTPEVTCVVVDVSHEDPEVKFNWYVD |
| | | GVEVHNAKTKPREEQYNSTYRVVSVLT |
| | | VLHQDWLNGKEYKCKVSNKALPAPIEK |
| | | TISKAKGQPREPQVYTLPPSREEMTKNQ |
| | | VSLTCLVKGFYPSDIAVEWESNGQPENN |
| | | YKTTPPVLDSDGSFFLYSKLTVDKSRWQ |
| | | QGNVFSCSVMHEALHNHYTQKSLSLSP |
| | | GK |
| 19 | mouse light chain | RADAAPTVSIFPPSSEQLTSGGASVVCFL |
| | constant region | NNFYPKDINVKWKIDGSERQNGVLNSW |
| | | TDQDSKDSTYSMSSTLTLTKDEYERHNS |
| | | YTCEATHKTSTSPIVKSFNRNEC |
| 20 | rabbit light chain | RADAAPTVSIFPPSSEQLTSGGASVVCFL |
| | constant region | NNFYPKDINVKWKIDGSERQNGVLNSW |
| | | TDQDSKDSTYSMSSTLTLTKDEYERHNS |
| | | YTCEATHKTSTSPIVKSFNRNEC |
| 21 | human light chain | RTVAAPSVFIFPPSDEQLKSGTASVVCLL |
| | constant region | NNFYPREAKVQWKVDNALQSGNSQES |
| | | VTEQDSKDSTYSLSSTLTLSKADYEKHK |
| | | VYACEVTHQGLSSPVTKSFNRGEC |

The anti-CLDN18.2 antibodies or antigen-binding fragments thereof provided herein can be a monoclonal antibody, a bispecific antibody, a multi-specific antibody, a recombinant antibody, a chimeric antibody, a humanized antibody, a labeled antibody, a bivalent antibody, an anti-idiotypic antibody, a fusion protein, a dimerized or polymerized antibody, or a modified antibody (e.g., glycosylated antibody). A recombinant antibody is an antibody prepared in vitro using recombinant methods rather than in animals.

In certain embodiments, the anti-CLDN18.2 antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

In some embodiments, the anti-CLDN18.2 antibodies and the antigen-binding fragments thereof provided herein comprise all or a portion of the heavy chain variable domain and/or all or a portion of the light chain variable domain. In one embodiment, the anti-CLDN18.2 antibody and the antigen-binding fragment thereof provided herein is a single domain antibody which consists of all or a portion of the heavy chain variable domain provided herein. More information of such a single domain antibody is available in the art (see, e.g., U.S. Pat. No. 6,248,516).

### Antibody Variants

The antibodies and antigen-binding fragments thereof provided herein also encompass various variants of the antibody sequences provided herein.

In certain embodiments, the antibody variants comprise one or more amino acid residue substitutions or modifications yet retains specific binding affinity to CLDN18.2. In certain embodiments, at least one of the substitutions or modifications is in one or more of the CDR sequences of the VH region or VL region. In certain embodiments, at least one of the substitutions or modifications is in one or more of the non-CDR sequences of the VH region or VL region. In certain embodiments, the antibodies or antigen-binding fragments thereof provided herein further comprise one or more non-natural amino acid (NNAA) substitutions. In certain embodiments, the NNAA is capable of being conjugated.

For example, the antibody variants comprise one or more amino acid residue substitutions or modifications in one or more of the CDR sequences provided in Table 1 above, one or more of the non-CDR sequences of the heavy chain variable region or light chain variable region provided in Table 2 above, and/or the constant region (e.g., Fc region). Such variants retain binding specificity to CLDN18.2 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, enhanced effector function(s), improved FcRn receptor binding, increased pharmacokinetic half-life, pH sensitivity, and/or compatibility to conjugation (e.g., one or more introduced cysteine residues), etc.

The parent antibody sequence may be screened to identify suitable or preferred residues to be modified or substituted, using methods known in the art, for example, “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

### a) Affinity Variants

Affinity variants of antibodies may contain modifications or substitutions in one or more CDR sequences provided in Table 1 above, one or more FR sequences, or the heavy or light chain variable region sequences provided in Table 2 above. FR sequences can be readily identified by a person skilled in the art based on the CDR sequences provided in Table 1 above and variable region sequences provided in Table 2 above, as it is well-known in the art that a CDR region is flanked by two FR regions in the variable region. The affinity variants retain specific binding affinity to CLDN18.2 of the parent antibody, or even have improved CLDN18.2 specific binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences, and variable region sequences, one or more amino acid residues may be substituted yet the resulting antibody or antigen-binding fragment still retain the binding affinity or binding capacity to CLDN18.2, or even have an improved binding affinity or capacity. Various methods known in the art can be used to achieve this purpose. For example, a library of antibody variants (such as Fab or scFv variants) can be generated and expressed with phage display technology, and then screened for the binding affinity to human CLDN18.2. For another example, computer software can be used to virtually simulate the binding of the antibodies to human CLDN18.2, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the anti-CLDN18.2 antibody or antigen-binding fragment thereof provided herein comprises 1, 2, or 3 CDR sequences having at least 80% (e.g., at least 85%, 86%, 87%, 88%, 89%, 90%, 91%, 92%, 93%, 94%, 95%, 96%, 97%, 98%, or 99%) sequence identity to that (or those) listed in Table 1 above yet retaining the specific binding affinity to CLDN18.2 at a level similar to or even higher than its parent antibody.

In certain embodiments, the anti-CLDN18.2 antibody or antigen-binding fragment thereof provided herein comprises one or more variable region sequences having at least 80% (e.g., at least 85%, 86%, 87%, 88%, 89%, 90%, 91%, 92%, 93%, 94%, 95%, 96%, 97%, 98%, or 99%) sequence identity to that (or those) listed in Table 2 above yet retaining the specific binding affinity to CLDN18.2 at a level similar to or even higher than its parent antibody. In some embodiments, a total of 1 to 10 amino acids have been substituted, inserted, or deleted in a variable region sequence listed in Table 2 above. In some embodiments, the substitutions, insertions, or deletions occur in regions outside the CDRs (e.g., in the FRs).

### b) Glycosylation Variants

The anti-CLDN18.2 antibody or antigen-binding fragment thereof provided herein also encompasses glycosylation variants, which can be obtained to either increase or decrease the extent of glycosylation of the antibodies or antigen-binding fragments thereof.

The antibodies or antigen-binding fragments thereof provided herein may comprise one or more modifications that introduce or remove a glycosylation site. A glycosylation site is an amino acid residue with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence is substituted. A new glycosylation site can be created in a similar way by introducing such a tripeptide sequence or serine or threonine residue.

### c) Cysteine-Engineered Variants

The anti-CLDN18.2 antibody or antigen-binding fragment thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### d) Fc Variants

The anti-CLDN18.2 antibody or antigen-binding fragment thereof provided herein also encompasses an Fc variant, which comprises one or more amino acid residue modifications or substitutions at its Fc region and/or hinge region.

In certain embodiments, the anti-CLDN18.2 antibody or antigen-binding fragment thereof provided herein comprises one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6 (1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70:3269-3277 (2010); and Hinton, P. et al., J. Immunology, 176:346-356 (2006).

In certain embodiments, the anti-CLDN18.2 antibody or antigen-binding fragment thereof provided herein comprises one or more amino acid substitution(s) that alters ADCC. Certain amino acid residues at CH2 domain of the Fc region can be substituted to provide for enhanced ADCC activity. Methods of altering ADCC activity by antibody engineering have been described in the art, see for example, Shields R L. et al., J Biol Chem. 2001. 276 (9): 6591-604; Idusogie E E. et al., J Immunol. 2000.164 (8): 4178-84; Steurer W. et al., J Immunol. 1995, 155 (3): 1165-74; Idusogie E E. et al., J Immunol. 2001, 166 (4): 2571-5; Lazar G A. et al., PNAS, 2006, 103 (11): 4005-4010; Ryan M C. et al., Mol. Cancer Ther., 2007, 6:3009-3018; Richards J O., et al., Mol Cancer Ther. 2008, 7 (8): 2517-27; Shields R. L. et al., J. Biol. Chem, 2002, 277:26733-26740; Shinkawa T. et al., J. Biol. Chem, 2003, 278:3466-3473.

In certain embodiments, the anti-CLDN18.2 antibody or antigen-binding fragment thereof comprises one or more amino acid substitution(s) that alters CDC, for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fc region variants. One or more amino acids selected from amino acid residues 329, 331 and 322 of the Fc region can be replaced with a different amino acid residue to alter C1q binding and/or enhance CDC (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-CLDN18.2 antibody or antigen-binding fragment thereof provided herein comprises one or more amino acid substitution(s) in human immunoglobulin (e.g., IgG1) at position 234 and/or 235 (according to EU numbering). In certain embodiments, the anti-CLDN18.2 antibody or antigen-binding fragment thereof provided herein comprises two amino acid substitutions in human immunoglobulin (e.g., IgG1) at positions 234 and 235 (according to EU numbering). In certain embodiments, the anti-CLDN18.2 antibody or antigen-binding fragment thereof provided herein comprises L234A and L235A (according to EU numbering) amino acid substitutions.

In certain embodiments, the anti-CLDN18.2 antibody or antigen-binding fragment thereof provided herein comprises one or more amino acid substitution(s) in human immunoglobulin (e.g., IgG4) at position 228 (according to EU numbering). In certain embodiments, the anti-CLDN18.2 antibody or antigen-binding fragment thereof provided herein comprises S228P (according to EU numbering) amino acid substitution.

In certain embodiments, the anti-CLDN18.2 antibody or antigen-binding fragment thereof provided herein comprises one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

In certain embodiments, the anti-CLDN18.2 antibody or antigen-binding fragment thereof provided herein comprises an amino acid substitution at position 366 (according to EU numbering) of a first Fc polypeptide, and comprises one, two or three amino acid substitutions at one, two or three positions of 366, 368, and 407 (according to EU numbering) of a second Fc polypeptide. In certain embodiments, the anti-CLDN18.2 antibody or antigen-binding fragment thereof provided herein comprises a T366W substitution (according to EU numbering) of a first Fc polypeptide, and comprise T366S+L368A+Y407V substitutions (according to EU numbering) of a second Fc polypeptide.

In certain embodiments, the anti-CLDN18.2 antibody or antigen-binding fragment thereof provided herein further comprises one or more amino acid substitution(s) of a first Fc polypeptide, and comprise one or more amino acid substitution(s) of a second Fc polypeptide, so as to introduce a non-natural disulfide bond between the two Fc polypeptides. For example, the anti-CLDN18.2 antibody or antigen-binding fragment thereof provided herein comprises an amino acid substitution at position 354 (according to EU numbering) of a first Fc polypeptide, and comprises an amino acid substitution at position 349 (according to EU numbering) of a second Fc polypeptide. In certain embodiments, the anti-CLDN18.2 antibody or antigen-binding fragment thereof provided herein comprises S354C substitution (according to EU numbering) of a first Fc polypeptide, and comprises Y349C substitution (according to EU numbering) of a second Fc polypeptide.

### Chimeric Antigen Receptor

In certain embodiments, the present disclosure provides a chimeric antigen receptor comprising the anti-CLDN18.2 antibody or an antigen-binding fragment thereof provided herein, a transmembrane region and an intracellular signal region.

The term “chimeric antigen receptor” or “CAR” or “CARs” as used herein refers to engineered receptors, which graft an antigen specificity onto cells (for example, T cells such as naive T cells, central memory T cells, effector memory T cells, regulatory T cells or combination thereof). CARs are also known as artificial T-cell receptors, chimeric T-cell receptors or chimeric immunoreceptors. In some embodiments, CARs comprise an antigen-specific targeting region (for example, the antigen-binding fragments of the anti-CLDN18.2 antibody as provided herein), an extracellular region, a transmembrane region, one or more co-stimulatory regions, and an intracellular signal region.

In some embodiments, the antigen-specific targeting region is an scFv. In some embodiments, the transmembrane region comprises a transmembrane region of CD3, CD4, CD8 or CD28. In some embodiments, the co-stimulatory region comprises a co-stimulatory domain of CD28, ICOS, CD27, 4-1BB, OX40 and CD40L. In some embodiments, the intracellular signal region is selected from the group consisting of: an intracellular signal region sequence of CD3 (e.g., CD3ζ), FcγRI, CD27, CD28, CD137, CD134, MyD88, CD40, CD278, TLRs, or a combination thereof.

The CARs may be grafted onto various cells, for example, allogeneic cells, autologous cells or xenogeneic cells.

The term “allogeneic cell” as used herein refers to any cells derived from a different subject of the same species.

The term “autologous cell” as used herein refers to any cells derived from the same subject into which they are later to be re-introduced.

The term “xenogeneic cell” as used herein refers to any cells derived from a different subject of a different species.

In some embodiments, the CARs are grafted on immune effector cells, for example, T cells, natural killer cells, macrophage cells, tumor-infiltrating lymphocytes, etc.

### Antibody-Based Assays and Techniques

Antibody-based assay and techniques are well developed for detection or diagnostic tests, by utilizing the highly specific interaction between the antigen and the antibody. An antigen could be proteins, peptides, polysaccharides or nucleic acids, which could be captured, detected or analyzed in antibody-based assays. In some embodiments, the antibody-based assay is immunohistochemistry (IHC), Immunocytochemistry (ICC), immunofluorescence (IF), enzyme immunoassay (EIA), Enzyme-Linked Immunosorbent Assay (ELISA), immunochromatography, or immunoblotting.

The term “immunohistochemistry (IHC)” as used herein refers to a method of selectively identifying antigens (proteins) in cells of a tissue section by immunostaining. Briefly, the selected antigen in a biological tissue sample, usually a fixed sample, could be mapped by staining with antibody.

The term “Immunocytochemistry (ICC)” as used herein refers to the method of anatomically visualizing the localization of a specific protein or antigen in cells by use of a specific primary antibody that binds to it. The primary antibody allows visualization of the protein under a fluorescence microscope when it is bound by a secondary antibody that has a conjugated fluorophore.

The term “immunofluorescence (IF)” as used herein refers to a histochemical staining technique used for demonstrating the presence of antibodies bound to antigens in tissues or serum. IF relies on the use of antibodies chemically labeled with fluorescent dyes to visualize molecules under a light microscope.

The terms “enzyme immunoassay (EIA)” or “Enzyme-Linked Immunosorbent Assay (ELISA)” as used herein refer to techniques that are based on the principle of immunoassay with an enzyme as the reporter label. In some embodiments, the antibody is fixed on a solid-phase support that could capture target the antigen in solution. In some embodiments, the capture antibody and detection antibody form a “sandwich” with the antigen. In some embodiments, an enzyme is covalently or non-covalently linked to the detection antibody, and the enzymatic reaction is conducted by adding substrate to develop color or chemiluminescence.

The term “immunochromatography” as used herein refers to a testing method for detecting a disease by dropping the sample containing an analyte onto a test strip. In some embodiment, test results of immunochromatography are judged by a visual check.

The term “immunoblotting” (i.e., Western blotting) as used herein refers to a qualitative and semiquantitative analysis of proteins that are separated by SDS-PAGE and transferred onto a membrane. Protein bands are probed by incubation with antibodies.

### Antigen-Binding Fragments

Provided herein are also anti-CLDN18.2 antigen-binding fragments. In some embodiments, the antibodies and antigen-binding fragments thereof provided herein comprise all or a portion of the heavy chain variable domain and/or all or a portion of the light chain variable domain. Various types of antigen-binding fragments are known in the art and can be developed based on the anti-CLDN18.2 antibodies provided herein, including for example, the exemplary antibodies whose CDR sequences are shown in Table 1 above, and their different variants (such as affinity variants, glycosylation variants, Fc variants, cysteine-engineered variants and so on).

In certain embodiments, an anti-CLDN18.2 antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multi-specific antibody, a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g., for Fab, Fv and ScFv antibody fragments), screening from a phage display library as discussed above (e.g., for scFv), and chemical coupling of two Fab′-SH fragments to form F(ab′) 2 fragments (Carter et al., Bio/Technology 10:163-167 (1992)). Other techniques for the production of antigen-binding fragments will be apparent to a person skilled in the art.

### Epitopes

In certain embodiments, the anti-CLDN18.2 antibody or an antigen-binding fragment thereof provided herein binds to an epitope within the amino acid sequence of SEQ ID NO: 9, 10, 11 or 12, or 13. The description and amino acid sequences of SEQ ID NO: 9~13 are shown in Table 10 below.

TABLE 10
CLDN18.2 peptide and fragment thereof

| SEQ ID | | | |
|---|---|---|---|
| NO: | Name | Description | Amino Acid Sequence |
| 9 | pTT5- | full length | MAVTACQGLGFVVSLIGIAGIIAATCM |
| | 022-A2 | human | DQWSTQDLYNNPVTAVFNYQGLWRS |
| | | CLDN18.2 | CVRESSGFTECRGYFTLLGLPAMLQA |
| | | protein | VRALMIVGIVLGAIGLLVSIFALKCIRI |
| | | | GSMEDSAKANMTLTSGIMFIVSGLCA |
| | | | IAGVSVFANMLVTNFWMSTANMYTG |
| | | | MGGMVQTVQTRYTFGAALFVGWVA |
| | | | GGLTLIGGVMMCIACRGLAPEETNYK |
| | | | AVSYHASGHSVAYKPGGFKASTGFGS |
| | | | NTKNKKIYDGGARTEDEVQSYPSKH |
| | | | DYV |
| 10 | 996659 | human | NNPVTAVFNYQGC |
| | | CLDN18.2 | |
| | | peptide | |
| | | fragment | |
| 11 | TC-20 | human | TQDLYNNPVTAVFNYQGLCC |
| | | CLDN18.2 | |
| | | peptide | |
| | | fragment | |
| 12 | YC-14 | human | YNNPVTAVFNYQGC |
| | | CLDN18.2 | |
| | | peptide | |
| | | fragment | |
| 13 | 996656 | human | TQDLYNNPVTAVFNYQGLWRSC |
| | | CLDN18.2 | |
| | | peptide | |
| | | fragment | |

The term “epitope” as used herein refers to the specific group of atoms or amino acids on an antigen to which an antibody binds. An epitope can be a conformational epitope or a linear epitope. In certain embodiments of the present disclosure, the epitopes bound by the anti-CLDN18.2 antibodies provided herein are linear. Those skilled in the art will recognize that it is possible to determine, without undue experimentation, if an antibody binds to the same or overlapping or adjacent epitope as the antibody of present disclosure (e.g., hybridoma/mouse antibody 43F11) by ascertaining whether the two competes for binding to a CLDN18.2 antigen polypeptide.

The present disclosure provides monoclonal antibodies directed against a certain epitope located within the N-terminal portion of CLDN18.2, which are useful in detecting and identifying cells expressing CLDN18.2, without cross-reacting to CLDN18.1. According to the sequences of human CLDN18.1 and CLDN18.2, there are 8 different amino acids located between amino acid 28-70 (i.e., N-terminal portion which comprises the first transmembrane (TM) region and loop 1), whereas the C-terminal sequences of human CLDN18.1 and CLDN18.2 are identical. The present disclosure for the first time provides monoclonal antibodies that bind to the N-terminal portion of CLDN18.2 with the epitope sequence as set forth in SEQ ID NO: 10, 11, 12, or 13.

In another aspect, the present disclosure provides monoclonal antibodies or antigen-binding fragments thereof, which competes for binding to CLDN18.2 with the antibody or antigen-binding fragment thereof provided herein, such as antibody 43F11.

The term “compete for binding” as used herein with respect to two antigen-binding proteins (e.g., antibodies), means that one antigen-binding protein blocks or reduces binding of the other to the antigen (e.g., human CLDN18.2) to any detectable degree, as determined by a competitive binding assay. Competitive binding assays are well known in the art, include, for example, direct or indirect radioimmunoassay (RIA), direct or indirect enzyme immunoassay (EIA), and sandwich competition assay (see, e.g., Stahli et al., 1983, Methods in Enzymology 9:242-253). Typically, such an assay involves the use of purified antigen bound to a solid surface or cells bearing the antigen, an unlabeled test antibody and a labeled reference antibody. Competitive inhibition is measured by determining the amount of label bound to the solid surface or cells in the presence of the test antibody. Usually, the test antibody is present in excess. If two antibodies compete for binding to the CLDN18.2, then the two antibodies bind to the same or overlapping epitope, or an adjacent epitope sufficiently proximal to the epitope bound by the other antibody for steric hindrance to occur. Usually, when a competing antibody is present in excess, it will inhibit (e.g., reduce) specific binding of a test antibody to a common antigen by at least 50-55%, 55-60%, 60-65%, 65-70%, 70-75% 75-80%, 80-85%, 85-90% or more.

### Polynucleotides and Recombinant Methods

The present disclosure provides isolated polynucleotides that encode the anti-CLDN18.2 antibodies or antigen-binding fragments thereof, and/or the chimeric antigen receptors provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the antibody or antigen-binding fragment thereof provided herein is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the antibodies or antigen-binding fragments thereof and/or the chimeric antigen receptors provided herein can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1α), and a transcription termination sequence.

The present disclosure provides vectors comprising the isolated polynucleotides provided herein. In certain embodiments, the polynucleotides provided herein encodes the antibodies or antigen-binding fragments thereof and/or the chimeric antigen receptors provided herein, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof and/or the chimeric antigen receptors provided herein can be introduced to a host expression system (e.g., a host cell) for cloning or gene expression. In certain embodiments, the host expression system provided herein is a microorganism, a yeast, or a mammalian cell. In certain embodiments, the microorganism is selected from the group consisting of E. coli and B. subtilis. In certain embodiments, the yeast is Saccharomyces. In certain embodiments, the mammalian cell is selected from the group consisting of COS, CHO—S, CHO-K1, HEK-293, and 3T3 cells.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-CLDN18.2 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; mouse forestomach carcinoma cells (MFC), SNU620 cells, and a human hepatoma line (Hep G2). In some embodiments, the host cell is a mammalian cultured cell line, such as CHO, BHK, NS0, 293, MFC, SNU620 and their derivatives.

Host cells are transformed with the above-described expression or cloning vectors for antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or antigen-binding fragment thereof and/or the chimeric antigen receptors provided herein, comprising culturing the host expression system provided herein under the condition at which the antibody or antigen-binding fragment thereof and/or the chimeric antigen receptor is expressed. The host expression systems used to produce the antibodies or antigen-binding fragments thereof and/or the chimeric antigen receptors provided herein may be cultured in a variety of media. Commercially available media such as Ham's F10 (Sigma), Minimal Essential Medium (MEM) (Sigma), RPMI-1640 (Sigma), and Dulbecco's Modified Eagle's Medium (DMEM) (Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibodies or antigen-binding fragments thereof and/or the chimeric antigen receptors prepared from the host expression systems can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof and/or the chimeric antigen receptors. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Conjugates

In some embodiments, the anti-CLDN18.2 antibodies or antigen-binding fragments thereof is linked or conjugated to one or more moieties. Examples of such moieties include but are not limited to, therapeutic agent (e.g., a DNA-alkylator, a topoisomerase inhibitor, a tubulin-binder, or other anti-cancer drugs), a detectable label, a pharmacokinetic modifying moiety (e.g., a polymer such as PEG which extends half-life), or a purifying moiety (e.g., a magnetic bead or a nanoparticle).

A moiety can be attached to the antibodies or antigen-binding fragments thereof either directly or via a linker or through another moiety, for example, by covalent binding, affinity binding, intercalation, coordinate binding, complexation, association, blending, or addition, among other methods. In certain embodiments, the antibodies or antigen binding fragments thereof are linked to one or more moieties via a linker. In certain embodiments, the linker is a hydrazone linker, a disulfide linker, a bifunctional linker, dipeptide linker, glucuronide linker, or a thioether linker.

In certain embodiments, the antibodies or antigen-binding fragments thereof provided herein may be engineered to contain specific sites outside the epitope binding portion that may be utilized for linking to one or more moieties. For example, such a site may include one or more reactive amino acid residues, such as for example cysteine or histidine residues, to facilitate covalent linkage to a moiety.

In some embodiments, the anti-CLDN18.2 antibodies or antigen-binding fragments thereof are linked to one or more moieties that function to (i) provide a detectable signal; (ii) interact with a second label to modify the detectable signal provided by the first or second label, e.g., FRET (Fluorescence Resonance Energy Transfer); (iii) affect mobility, e.g., electrophoretic mobility, by charge, hydrophobicity, shape, or other physical parameters, or (iv) provide a capture moiety, e.g., affinity, antibody/antigen, or ionic complexation.

Such moieties include, but are not limited to, labels or moieties that are directly detectable (such as radioactive isotope, a lanthanide, a chemiluminescent label, a chromophoric moiety, an enzyme label, colloidal gold particles and a fluorescent label) via imaging, an enzymatic reaction and so on, as well as moieties that are indirectly detectable, for example, through a molecular interaction. Examples of indirectly detectable label include, biotin/avidin, biotin/streptavidin, a digoxigenin label, a hapten, a DNA molecule for detection and particle labels (paramagnetic particle, metal particle labels, magnetic particle labels, and polymer particle labels, etc.).

Examples of radioactive isotopes include such as S, C, I, H, and I. The antibody can be labeled with the radioisotope using the techniques described in, for example, Current Protocols in Immunology, Volumes 1 and 2, Coligen et al., Ed. Wiley-Interscience, New York, N.Y., Pubs. (1991), and radioactivity can be measured using scintillation counting. Other radionuclides include ¹²³I, ¹²⁴I, ¹²⁵I, ¹³¹I, ³⁵S, ³H, ⁹⁹Tc, ⁹⁰Y, ¹¹¹In, ¹¹²In, ³²P, ¹⁴C, ¹⁵O, ¹³N, ¹⁸F, ⁸⁶Y, ⁸⁸Y, ⁹⁰Y, ⁵¹Cr, ⁵⁷To, ²²⁵Ra, ⁶⁰Co, ⁵⁹Fe, ⁵⁷Se, ¹⁵²Eu, ⁶⁴Cu, ⁶⁷Cu, ¹⁷⁷Lu, ²¹¹At, ¹⁸⁶Re, ¹⁸⁸Re, ¹⁵³Sm, ²¹²Bi, ²¹²Pb, ⁴⁷Sc, ¹⁰⁹Pd, ²³⁴Th, ⁴⁰K, ¹⁵⁷Gd, ⁵⁵Mn, ⁵²Tr, or ⁵⁶Fe.

Fluorescent or luminescent labels include, but not limited to, rare earth chelates (europium chelates), fluorescein and its derivatives, rhodamine and its derivatives, isothiocyanate, phycoerythrin, phycocyanin, allophycocyanin, o-phthaladehyde, fluorescamine, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, Texas Red, dansyl, Lissamine, umbelliferone, phycocrytherin, phycocyanin, or commercially available fluorophores such SPECTRUM ORANGE® and SPECTRUM GREEN® and/or derivatives of any one or more of the above. The fluorescent labels can be conjugated to the antibody using the techniques disclosed in, for example, Current Protocols in Immunology, supra, for example. Fluorescence can be quantified using a fluorimeter.

Another type of useful label is enzyme-substrate label. Various enzyme-substrate labels are available (see U.S. Pat. No. 4,275,149). The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor.

Numerous enzyme-substrate combinations are available to those skilled in the art (see U.S. Pat. Nos. 4,275,149 and 4,318,980), such as: (i) Horseradish peroxidase (HRP) with hydrogen peroxidase as a substrate, wherein the hydrogen peroxidase oxidizes a dye precursor, such as, e.g., 3,3′ diamino benzidine (DAB), which produces a brown end product; 3-amino-9-ethylcarbazole (AEC), which upon oxidation forms a rose-red end product; 4-chloro-l-napthol (CN), which precipitates as a blue end product; and p-Phenylenediamine dihydrochloride/pyrocatecol, which generates a blue-black product; orthophenylene diamine (OPD) and 3,3′,5,5′-tetramethyl benzidine hydrochloride (TMB); (ii) alkaline phosphatase (AP) and para-Nitrophenyl phosphate, naphthol AS-MX phosphate, Fast Red TR and Fast Blue BB, napthol AS-BI phosphate, napthol AS-TR phosphate, 5-bromo-4-chloro-3-indoxyl phosphate (BCIP), Fast Red LB, Fast Garnet GBC, Nitro Blue Tetrazolium (NBT), and iodonitrotetrazolium violet (INT); and (iii) β-D-galactosidase (β-D-Gal) with a chromogenic substrate (e.g., p-nitrophenyl-P-D-galactosidase) or fluorogenic substrate (e.g., 4-methylumbelliferyl-P-D-galactosidase).

Other useful enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al, Methods for the Preparation of Enzyme-Antibody Conjugates for use in Enzyme Immunoassay, in Methods in Enzym. (ed J. Langbne & H. Van Vunakis), Academic press, New York, 73:147-166 (1981).

### Pharmaceutical Compositions

The present disclosure further provides pharmaceutical compositions comprising the antibodies or antigen-binding fragments thereof and/or the chimeric antigen receptors provided herein and one or more pharmaceutically acceptable carriers.

Pharmaceutical acceptable carriers for use in the pharmaceutical compositions disclosed herein may include, for example, pharmaceutically acceptable liquid, gels, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agents. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Kits

In certain embodiments, the present disclosure provides kits comprising the isolated antibody or antigen-binding fragment thereof provided herein. In certain embodiments, the kit disclosed herein is a diagnostic kit. The kits may be useful in detection of presence or amount of CLDN18.2 in a biological sample, or may be useful in the methods of diagnosis provided herein.

In certain embodiments, the kit comprises the antibody or the antigen binding fragment thereof provided herein which is optionally detectably labeled. In certain embodiments, the antibody or the antigen binding fragment thereof is conjugated with an indirectly detectable moiety. In certain embodiments, the kit further comprises a second agent. in certain embodiments, the second agent is a diagnostic agent.

In certain embodiments, the kit further comprises a set of reagents for detecting a complex of the antibody or antigen-binding fragment thereof bound to CLDN18.2, useful in a variety of detection assays, including for example, immunoassays such as IHC, ICC, or ELISA (sandwich-type or competitive format).

In yet another embodiment of the invention, the reagents useful to perform immunohistochemistry on an FFPE tumor tissue section are provided in a kit along with instructions for performing the IHC assay.

Any of the indirectly detectable labels or moieties disclosed herein can be used. In certain embodiments, the indirectly detectable moiety comprises biotin. In such embodiments, the set of reagents comprises a detectably labeled avidin or steptavidin.

In certain embodiments, a detectable label is conjugated to the antibody or antigen-binding fragment thereof provided herein. In certain embodiments, the kits comprise the isolated antibody or antigen-binding fragment thereof provided herein and a secondary antibody that is conjugated with a detectable label. In some embodiments, the secondary antibody comprises an antibody that specifically binds to the primary antibody (e.g., the antibody or antigen-binding fragment thereof provided herein). In some embodiments, the secondary antibody can be an anti-mouse antibody, an anti-rabbit antibody, or anti-human antibody.

The detectable label may require combination with one or more components, e.g., buffers, antibody-enzyme conjugates, enzyme substrates, or the like, prior to use, and such reagents can be included in the kits. For example, when the detectable moiety comprises an enzyme, the kit will include substrates and co factors required by the enzyme (e.g., a substrate precursor which provides the detectable chromophore or fluorophore). In addition, other reagents may be included such as blocking reagents for reducing nonspecific binding to the solid phase surface, washing reagents, enzyme substrates, and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration. Instructions, either as inserts or a label, indicating guidelines for detection and/or assay system preparation, can also be included in the kit.

The kit's components may be pre-attached to a solid support, or may be applied to the surface of a solid support when the kit is used. The solid phase surface may be in the form of a tube, a bead, a microtiter plate, a microsphere, or other materials suitable for immobilizing proteins, peptides, or polypeptides.

Containers for use in such kits may typically comprise at least one vial, test tube, flask, bottle, syringe or other suitable container, into which one or more of the detection composition(s) may be placed, and preferably suitably aliquoted. The kits disclosed herein will also typically include a means for containing the vial(s) in close confinement for commercial sale, such as, e.g., injection or blow-molded plastic containers into which the desired vial(s) are retained. Where a radiolabel, chromogenic, fluorigenic, or other type of detectable label or detecting means is included within the kit, the labeling agent may be provided either in the same container as the detection composition itself or may alternatively be placed in a second distinct container means into which this second composition may be placed and suitably aliquoted. Alternatively, the detection reagent may be prepared in a single container means, and in most cases, the kit will also typically include a means for containing the vial(s) in close confinement for commercial sale and/or convenient packaging and delivery.

A device or apparatus for carrying out the detection or monitoring methods described herein is also provided. Such an apparatus may include a chamber or tube into which sample can be input, a fluid handling system optionally including valves or pumps to direct flow of the sample through the device, optionally filters to separate plasma or serum from blood, mixing chambers for the addition of capture agents or detection reagents, and optionally a detection device for detecting the amount of detectable label bound to the capture agent immunocomplex. The flow of sample may be passive (e.g., by capillary, hydrostatic, or other forces that do not require further manipulation of the device once sample is applied) or active (e.g., by application of force generated via mechanical pumps, electroosmotic pumps, centrifugal force, or increased air pressure), or by a combination of active and passive forces.

### Detection of CLDN18.2 and Methods of Use

In another aspect, the present disclosure provides a method of detecting presence or expression level of CLDN18.2 in a sample, comprising contacting the sample with the antibody or antigen-binding fragment thereof under a condition that allows specific binding of the antibody or antigen-binding fragment thereof to CLDN18.2, and determining presence or expression level of CLDN18.2 in the sample.

In another aspect, the present disclosure provides a method for diagnosing a CLDN18.2-associated disease, disorder or condition (e.g., cancer), screening for the onset or risk to the onset of a CLDN18.2-associated disease, disorder or condition (e.g., cancer), or assessing the development or prognosis of a CLDN18.2-associated disease, disorder or condition (e.g., cancer) in a subject, comprising:
a) contacting a sample obtained from the subject with the antibody or antigen-binding fragment thereof or the chimeric antigen receptor provided herein under a condition that allows specific binding of the antibody or antigen-binding fragment thereof or the chimeric antigen receptor to CLDN18.2; and
b) determining presence or expression level of CLDN18.2 in the sample; wherein the subject is diagnosed as having a CLDN18.2-associated disease, disorder or condition (e.g., cancer), or is at the onset or at a risk to the onset of a CLDN18.2-associated disease, disorder or condition (e.g., cancer), or develops or with an increased probability of developing a CLDN18.2-associated disease, disorder or condition (e.g., cancer), or has poor prognosis or at a risk to poor prognosis of a CLDN18.2-associated disease, disorder or condition (e.g., cancer) when the presence of CLDN18.2 is found or when the expression level of CLDN18.2 equals to or is higher than a threshold level.

In another aspect, the present disclosure provides a method for determining the eligibility of a subject having or at risk of having a CLDN18.2-associated disease, disorder or condition for treatment with a CLDN18.2-targeting agent, comprising:
a) contacting a sample obtained from the subject with the antibody or antigen-binding fragment thereof or the chimeric antigen receptor provided herein under a condition that allows specific binding of the antibody or antigen-binding fragment thereof or the chimeric antigen receptor to CLDN18.2; and
b) determining presence or expression level of CLDN18.2 in the sample;
   wherein the subject is determined as eligible for treatment with a CLDN18.2-targeting agent when the presence of CLDN18.2 is found or when the expression level of CLDN18.2 equals to or is higher than a threshold level, or
   wherein the subject is determined as not eligible for treatment with a CLDN18.2-targeting agent when the presence of CLDN18.2 is not found or when the expression level of CLDN18.2 is lower than a threshold level.

In another aspect, the present disclosure provides a method of predicting therapeutic effectiveness of a CLDN18.2-targeting agent in treating a CLDN18.2-associated disease, disorder or condition in a subject, comprising:
a) contacting a sample obtained from the subject with the antibody or antigen-binding fragment thereof or the chimeric antigen receptor provided herein under a condition that allows specific binding of the antibody or antigen-binding fragment thereof or the chimeric antigen receptor to CLDN18.2;
b) determining presence or expression level of CLDN18.2 in the sample; and
c) predicting the therapeutic effectiveness of the CLDN18.2-targeting agent,
   wherein the CLDN18.2-targeting agent is predicted to be effective in treating the subject when the presence of CLDN18.2 is found or when the expression level of CLDN18.2 equals to or is higher than a threshold level, or
   wherein the CLDN18.2-targeting agent is predicted to be not effective in treating the subject when the presence of CLDN18.2 is not found or when the expression level of CLDN18.2 is lower than the threshold level.

In another aspect, the present disclosure provides a method of treating, preventing or alleviating a CLDN18.2-associated disease, disorder or condition in a subject having or at risk of having the CLDN18.2-associated disease, disorder or condition, comprising:
a) selecting a subject that is suitable for the treatment, prevention or alleviation, comprising:
   (i). contacting a sample obtained from the subject with the antibody or antigen-binding fragment thereof or the chimeric antigen receptor provided herein under a condition that allows specific binding of the antibody or antigen-binding fragment thereof or the chimeric antigen receptor to CLDN18.2;
   (ii). determining the presence or expression level of CLDN18.2 in the sample; and
   (iii). selecting the subject as suitable for the treatment, prevention or alleviation of the CLDN18.2-associated disease, disorder or condition when the presence of CLDN18.2 is found or when the expression level of CLDN18.2 in the sample equals to or is higher than a threshold level;
b) administering a therapeutically effective amount of CLDN18.2-targeting agent to the selected subject.

In another aspect, the present disclosure provides a method of treating, preventing or alleviating a CLDN18.2-associated disease, disorder or condition in a subject having or at risk of having the CLDN18.2-associated disease, disorder or condition, comprising:
a) selecting a subject that is suitable for the treatment, prevention or alleviation, comprising:
   (i). contacting a sample obtained from the subject with the antibody or antigen-binding fragment thereof or the chimeric antigen receptor provided herein under a condition that allows specific binding of the antibody or antigen-binding fragment thereof or the chimeric antigen receptor to CLDN18.2;
   (ii). determining the presence or expression level of CLDN18.2 in the sample; and
   (iii). selecting the subject as not suitable for the treatment, prevention or alleviation of the CLDN18.2-associated disease, disorder or condition with a CLDN18.2-targeting agent when the presence of CLDN18.2 is not found or when the expression level of CLDN18.2 in the sample is lower than a threshold level;
b) administering to the selected subject a Standard-of-Care therapeutic other than a CLDN18.2-targeting agent.

In certain embodiments, the sample is selected from the group consisting of a cell sample, a tissue sample, biopsy, a paraffin-embedded tissue, a body fluid (e.g., whole blood, blood serum, blood plasma, urine, mucus, saliva, peritoneal fluid, pleural fluid, chest fluid, synovial fluid, cerebrospinal fluid, thoracentesis fluid, abdominal fluid, and any combination thereof), colonic effluent, a surgical resection sample, an isolated blood cell, a cell isolated from blood, and any combination thereof.

Any biological sample suspected of containing CLDN18.2 can be detected in the methods provided herein. In some embodiments, a suitable sample can be a cell sample or a tissue sample obtained from a subject in need of the detection. For example, the sample may include, normal and cancerous tissue of stomach, lung, breast, colon, kidney, bone, brain, muscle, pancreas, bladder, ovary, uterus, as well as heart, embryonic, or placental tissue. Preferably a sample contains cells or tissue of the organ which is to be examined, e.g., which is to be diagnosed for cancer. For example, if the cancer to be diagnosed is gastric cancer a sample may contain cells or tissue obtained from stomach. In some embodiments, the sample is a tumor sample. In certain embodiments, the tissue samples are samples with CLDN18.2-associated disease or condition.

In certain embodiments, the cell sample is produced from a cell block. A “cell block” is a method of preparing cytologic material so that it can be processed, sectioned, stained, and viewed as a histology section. It can provide diagnostic information in addition to that obtained from cytology slides. In certain embodiments, cell blocks can be prepared from residual effusions, sputum, urine sediments, gastrointestinal fluids, cell scraping, or fine needle aspirates. Cells are concentrated or packed by centrifugation or membrane filtration.

In some embodiments the sample can contain for example, preservatives, anticoagulants, buffers, nutrients, antibiotics, or the like. In certain embodiments, the sample has been exposed to and/or contains one or more fixatives. Exemplary fixatives suitable for the methods provided herein include formalin, glutaraldehyde, osmium tetraoxide, acetic acid, ethanol, acetone, picric acid, chloroform, potassium dichromate and mercuric chloride and/or stabilizing by microwave heating or freezing.

In some embodiments, the sample comprises a fixed tissue sample. In some embodiments, the fixed tissue sample is a formalin-fixed paraffin-embedded (FFPE) tissue. FFPE tissue sections can be of about 3-4 millimeters, and preferably 4-40 micrometers, which are mounted and dried on a microscope slide. Examples of paraffin include, but are not limited to, Paraplast, Broloid and Tissuemay. For a fixed tissue sample such as an FFPE tissue sample, the sample may be deparaffinized before contacting with the anti-CLDN18.2 antibody or antigen-binding fragment thereof provided herein.

In certain embodiments, the presence or expression level of cell surface or membrane-bound CLDN18.2 is detected or determined in the methods provided herein. The phrase “cell surface or membrane-bound CLDN18.2” means that CLDN18.2 is associated with and located at the plasma membrane of a cell, wherein at least a part of the CLDN18.2 is exposed to the extracellular space of said cell and is accessible from the outside of said cell, e.g., by antibodies outside the cell. In certain embodiments, the part of the CLDN18.2 that is exposed extracellularly comprises at least 4, at least 8, at least 10, at least 12, or at least 20 amino acid residues. In normal tissues except stomach epithelial cells, CLDN18.2 are located within the tight junctions of epithelia and endothelia, and are believed to be not accessible to antibodies from outside the cells, and thus considered not cell surface or membrane-bound CLDN18.2.

The presence or expression level of CLDN18.2 protein in a sample can be determined based on the presence or level of the complex of the CLDN18.2 antigen bound by the antibody, or the antigen binding fragment thereof disclosed herein. Any suitable methods can be used for detection of the antibody-antigen complex, for example, by immunoassays such as immunohistochemistry (IHC), Immunocytochemistry (ICC), immunofluorescence (IF), immunoblotting (e.g., Western blotting), flow cytometry (e.g., FACS™), Enzyme-linked Immunosorbant Assay (ELISA), enzyme immunoassay (EIA), and radioimmunoassay (RIA). For a review of immunological and immunoassay procedures, see Basic and Clinical Immunology (Stites & Terr eds., 7th ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Terr, eds., 7th ed. 1991).

To allow direct detection of the antigen (e.g., CLDN18.2), one can use the anti-CLDN18.2 antibodies or the antigen binding fragments thereof disclosed herein that are linked to a detectable label, which allows direct visualization without the need of further antibody interaction.

The presence of CLDN18.2 expression in the sample can be confirmed by presence of positively-stained cell, for example, a cell having at least partial membrane staining of any intensity for the anti-CLDN18.2 antibody staining. In certain embodiments, a normal sample can be used as a control sample, and the presence of CLDN18.2 expression in the test sample can be determined relative to the control sample. The term “normal” such as used in the term “normal sample” or “normal tissue” refers to a sample or a tissue from a healthy or non-cancerous subject, or a sample or tissue from a healthy or non-cancerous tissue. Preferably, the test and control samples are comparable in terms of type of sample, for example, both are fixed tissue sample. If the test sample shows an increase in staining intensity or in number of positive stained cells than the control sample, then the test sample can be determined as positive for CLDN18.2 expression.

In certain embodiments, the expression level of CLDN18.2 can be quantified using any suitable methods known in the art, for example, by determination of the relative proportion of positively-stained cells and the staining intensity on the cell membrane.

In certain embodiments, the CLDN18.2 expression level is quantified based on the percentage of positively-stained cells (i.e., CLDN18.2-positive cells) in the sample. A CLDN18.2-positive cell is cell having at least partial membrane staining of any intensity for the anti-CLDN18.2 antibody staining. For example, to assess CLDN18.2 expression in a sample, an observer can examine the number of membrane CLDN18.2+ cells in one or more selected field(s) under a microscope and calculate or estimate the percentage of cells that are positive for CLDN18.2. If the sample is highly heterogeneous, then the sample can be divided into zones, and each zone is scored separately and then combined into a single set of percentage values.

In certain embodiments, the expression level is quantified based on staining intensity for CLDN18.2 (e.g., membrane-bound CLDN18.2) in the sample. For example, an intensity score such as 4-point HSCORE can be calculated based on intensity of staining ranging from 0 (no staining), 1+ (weak staining), 2+ (distinct staining), 3+ (strong staining) and 4+ (extremely strong/saturated signal) and multiplying by the percent of cells staining at each intensity (0 to 100%) (see details in, McCarty, K. S. Jr, et al, Cancer Res. 46 (suppl 8): 4244s-4248s (1986)). For another example, Alfred score can be calculated based on a Total Score (TS, range 0 to 8) by adding together a proportion score (PS) and an intensity score (IS). PS is the proportion of positive tumor cells ranging from 0 to 5 (0=no positive cells, 1=1/100 cells are positive, 2=1/10 cells are positive, 3=1/3 cells are positive, 4=2/3 of cells are positive, 5=all tumor cells are positive). IS means the average staining intensity of positive tumor cells ranging from 0 to 3 (0=negative, 1=weak, 2=intermediate staining, 3=strong staining) (see, details in, Alfred D C et al. Mod Pathol. 11:155-168 (1998)).

In certain embodiments, the samples are assessed by two observers operating independently and the percentage or the scores are subsequently consolidated. In certain other embodiments, the identification of positive and negative cells is performed or scored using appropriate software.

In certain embodiments, the level of the CLDN18.2 can be determined, for example, by normalizing to a control value or to a standard curve. The control value can be predetermined, or determined concurrently, from a negative control sample or a blank control sample.

In certain embodiments, for the diagnostic or clinical applications, the expression level of CLDN18.2 (e.g., exposed on the cell surface) in the test sample is compared to a threshold level.

A “threshold level” or “threshold value” with respect to CLDN18.2 expression, refers to a level of expression that allows for distinguish of being positive from being negative for cell surface CLDN18.2 expression, or a level of expression that allows for ruling in or ruling out of a CLDN18.2-associated condition, for example, cancer, or the onset or risk to the onset of cancer in a subject, or a threshold level which allows for monitoring treatment response in a subject who is receiving treatment of cancer. In certain embodiments, the threshold is determined relative to a control expression level.

For example, the threshold can be a level above which the sample is scored as having positive expression of CLDN18.2, and hence eligibility for treatment with a CLDN18.2-targeting agent. If the level of CLDN18.2 in the sample reaches or is above the threshold, it could indicate presence of the CLDN18.2-associated disease or condition, and/or likelihood of responding to a CLDN18.2-targeting agent.

The threshold level can be determined by a skilled person in the art, taking consideration of a variety of factors, including for example, the type of sample, the detection method, the disease or condition to be diagnosed, and/or the CLDN18.2-targeting agents to be used.

In certain embodiments, the presence of CLDN18.2 or CLDN18.2-expressing cells and/or a quantity of CLDN18.2 or CLDN18.2-expressing cells which is increased compared to a threshold level, e.g., compared to a subject without a CLDN18.2-associated condition (e.g., a non-cancerous subject), indicates the presence of or risk for (i.e., a potential for a development of) CLDN18.2-associated disease or condition (e.g., a cancer disease) in the patient.

In certain embodiments, the threshold level is a percentage of CLDN18.2 positive cells (%) that have at least partial membrane staining of any intensity. In one embodiment, the threshold level of percentage of CLDN18.2 positive cells (%) is 1%, 2%, 5%, 10%, 15%, 20%, 25%, 30%, 35%, 40%, 45%, 50%, 55%, 60%, 65%, or 70%.

In certain embodiments, the sample is from a subject having, or at risk of, a CLDN18.2-associated disease or condition. The term “CLDN18.2-associated disease or condition” as used herein refers a disease or condition characterized in having an increased expression of CLDN18.2 in cells of a diseased tissue or organ compared to the state in a healthy or noncancerous tissue or organ other than stomach. An increase can be for example an increase by at least 10%, 20%, 30%, 40%, 50%, 100%, 200%, 300%, 400%, 500%, or even more. In certain embodiments, the expression of CLDN18.2 in cells of a diseased tissue or organ is above the detection limit and/or is high enough to allow binding by CLDN18.2-specific antibodies added to the cells. In some embodiments, increase in expression of CLDN18.2 is only found in a diseased tissue, while the expression of CLDN18.2 in normal tissue is not detectable.

In some embodiments, the CLDN18.2-associated disease or condition is characterized in increased expression in cell surface or membrane-bound CLDN18.2.

In some embodiment, the CLDN18.2-related disease or condition is cancer. In one embodiment, cancer cells express or aberrantly express CLDN18.2 while the corresponding normal cells do not express CLDN18.2 or express CLDN18.2 at a lower level. It is believed that CLDN18.2 as a tight junction protein may be a good therapeutic target for CLDN18.2-associated disease such as tumor, and hence can be used to select patients eligible for treatment with CLDN18.2-targeting agents. Unlike normal epithelial tissue (except stomach epithelial cells) in which CLDNs associate to form classical tight junctions, CLDNs expressed in tumor cells often do not form such classical tight junctions, and as a result, tumor cells are likely to have free CLDNs that are exposed and accessible to extracellular antibody binding and immunotherapy.

CLDN18.2 is a valuable target for the prevention and/or treatment of primary tumors, such as gastric cancer, lung cancer (e.g., non-small cell lung cancer (NSCLC, squamous/nonsquamous), small cell lung cancer (SCLC)), bronchial cancer, bone cancer, liver and bile duct cancer, pancreatic cancer, breast cancer (including basal breast carcinoma, ductal carcinoma and lobular breast carcinoma), liver cancer, ovarian cancer, testicle cancer, kidney cancer, bladder cancer, head and neck cancer, spine cancer, brain cancer, cervix cancer, uterine cancer, endometrial cancer, hepatic cancer, head-neck cancer, cancer of gallbladder, colon cancer, colorectal cancer, rectal cancer, anal cancer, esophageal cancer, gastrointestinal cancer, skin cancer, prostate cancer, pituitary cancer, stomach cancer, vagina cancer, thyroid cancer, glioblastoma, astrocytoma, melanoma, myelodysplastic syndrome, sarcoma, teratoma, cholangiocarcinoma, and/or adenocarcinoma, and/or metastases thereof, in particular gastric cancer metastasis such as Krukenberg tumors, peritoneal metastasis, and lymph node metastasis.

In certain embodiments, the cancer is primary cancer or metastatic cancer.

In certain embodiments, the cancer includes but are not limited to, renal cell cancer, gastric carcinoma, mesothelioma, melanoma, cervical cancer, thymic carcinoma, myelomas, mycoses fungoids, merkel cell cancer, hepatocellular carcinoma (HCC), fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, lymphoid malignancy, basal cell carcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, classical Hodgkin lymphoma (CHL), primary mediastinal large B-cell lymphoma, T-cell/histiocyte-rich B-cell lymphoma, acute lymphocytic leukemia, acute myelocytic leukemia, acute myelogenous leukemia, chronic myelocytic (granulocytic) leukemia, chronic myelogenous leukemia, chronic lymphocytic leukemia, polycythemia vera, mast cell derived tumors, EBV-positive and -negative PTLD, and diffuse large B-cell lymphoma (DLBCL), plasmablastic lymphoma, extranodal NK/T-cell lymphoma, nasopharyngeal carcinoma, HHV8-associated primary effusion lymphoma, non-Hodgkin's lymphoma, multiple myeloma, Waldenstrom's macroglobulinemia, heavy chain disease, myelodysplastic syndrome, hairy cell leukemia and myelodysplasia, primary CNS lymphoma, spinal axis tumor, brain stem glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma.

In certain embodiments, the cancer is gastric cancer, colon cancer, hepatocellular carcinoma, ovarian cancer, cholangiocarcinoma, pancreatic cancer, breast cancer, lung cancer (e.g., non-small cell lung cancer or small cell lung cancer), bronchial cancer, bone cancer, liver and bile duct cancer, liver cancer, testicle cancer, kidney cancer, bladder cancer, head and neck cancer, spine cancer, brain cancer, cervix cancer, uterine cancer, endometrial cancer, colorectal cancer, rectal cancer, anal cancer, esophageal cancer, gastrointestinal cancer, skin cancer, prostate cancer, pituitary cancer, vagina cancer, thyroid cancer, glioblastoma, astrocytoma, melanoma, myelodysplastic syndrome, sarcoma, teratoma, or adenocarcinoma.

In certain embodiments, the cancer is gastric cancer, colon cancer, hepatocellular carcinoma, ovarian cancer, cholangiocarcinoma, pancreatic cancer, breast cancer, or lung cancer (e.g., non-small cell lung cancer or small cell lung cancer).

In certain embodiments, the cancer is a CLDN18.2-expressing cancer. The presence or expression level of CLDN18.2 in the sample indicates whether cancer cells express CLDN18.2, and hence whether the cancer is likely to respond to treatment with a CLDN18.2-targeting agent.

“CLDN18.2-targeting agent” as used herein refers to one or more agents that target CLDN18.2 protein or nucleic acids (DNA or mRNA) in a cell, a tissue or a living body. A CLDN18.2-targeting agent may decrease/eliminate the expression of CLDN18.2 gene product, inhibit/disrupt the signaling of CLDN18.2, or induce cytotoxicity to cells that abnormally express CLDN18.2.

In certain embodiments, the CLDN18.2-targeting agent includes a therapeutic anti-CLDN18.2 antibody, a CLDN18.2-binding molecule, a cell therapy targeting CLDN18.2, a chemical compound targeting CLDN18.2, or a therapeutic nucleic acid targeting CLDN18.2. In certain embodiments, the CLDN18.2-targeting agent is capable of inducing cytotoxicity to CLDN18.2-expressing cells.

In certain embodiments, the CLDN18.2-targeting agent comprise a therapeutic anti-CLDN18.2 antibody or a CLDN18.2-binding molecule. In certain embodiments, the therapeutic anti-CLDN18.2 antibody or a CLDN18.2-binding molecule can induce ADCC, CDC or ADCP to the CLDN18.2-expressing cells. Alternatively, the therapeutic anti-CLDN18.2 antibody or a CLDN18.2-binding molecule can be conjugated to a cytotoxic agent, for example, to form an antibody-drug conjugate (ADC). In certain embodiments, the cytotoxic agent can be any agent that is detrimental to cells or that can damage or kill cells. In certain embodiments, the cytotoxic agent is optionally a toxin, a chemotherapeutic agent (such as a DNA-alkylators, a topoisomerase inhibitor, a tubulin-binders, a growth inhibitory agent, or other anticancer drugs), or a radioactive isotope. In other embodiments, the therapeutic anti-CLDN18.2 antibody can be a bispecific antibody that binds to different antigens or different epitopes on the CLDN18.2 protein.

In certain embodiments, the CLDN18.2-targeting agent comprise a CLDN18.2-targeting cell therapy. In certain embodiments, the CLDN18.2-targeting cell therapy includes a CAR-T (Chimeric Antibody Receptor Engineered T Cell), TCR-T (Gene Modified TCR T cells) or CAR-NK (Chimeric Antibody Receptor Engineered NK Cell) expressing a CLDN18.2-binding Chimeric Antibody Receptor (CAR). Chimeric antigen receptors (CARs) are engineered chimeric receptors that combine an antigen-binding domain of an antibody with one or more signaling domains for T cell activation. Immune cells such as T cells and Nature Killer (NK) cells can be genetically engineered to express CARs or genetically modified TCRs (see, for details, D. Li et al., Sig Transduct Target Ther 4, 35 (2019), S. Kloess et al., Transfus Med Hemother; 46:4-13 (2019), Wang W. et al., Cancer Letters, 472:175-180 (2020)). T cells expressing a CAR are referred to as CAR-T cells. CAR can mediate antigen-specific cellular immune activity in the T cells, enabling the CAR-T cells to eliminate cells (e.g., tumor cells) expressing the targeted antigen. In one embodiment, binding of the CAR-T cells provided herein to CLDN18.2 expressed on cells such as cancer cells, results in proliferation and/or activation of said CAR-T cells, wherein said activated CAT-T cells can release cytotoxic factors, e.g., perforin, granzymes, and granulysin, and initiate cytolysis and/or apoptosis of the cancer cells.

In certain embodiments, the therapeutic nucleic acid targeting CLDN18.2 can be short interfering nucleic acid (siNA), short interfering RNA (siRNA), double-stranded RNA (dsRNA), micro-RNA (miRNA), and short hairpin RNA (shRNA) molecules capable of mediating RNA interference (RNAi) against CLDN18.2 gene sequence, or another gene sequence in the CLDN18.2-expressing cell.

In certain embodiments, a CLDN18.2-targeting agent promotes cancer regression in a subject. In preferred embodiments, a therapeutically effective amount of the CLDN18.2-targeting agent promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction.

In certain embodiments, the subject is receiving or has received anti-cancer-therapy, or suffers from cancer recurrence. An anti-cancer-therapy includes, for example, but not limited to, a chemotherapeutic agent, an anti-cancer drug, radiation therapy, an immunotherapy, anti-angiogenesis agent, a targeted therapy, a cellular therapy, a gene therapy, a hormonal therapy, palliative care, surgery for the treatment of cancer (e.g., tumorectomy), or one or more anti-emetics or other treatments for complications arising from chemotherapy.

A relapse or recurrence occurs when a person is affected again by a condition that affected them in the past. For example, if a patient has suffered from a cancer disease, has received a successful treatment of said disease and again develops said disease. Said newly developed disease may be considered as relapse or recurrence. However, according to the present disclosure, a relapse or recurrence of a cancer disease may, but does not necessarily, occur at the site of the original cancer disease. Thus, for example, if a patient has suffered from gastric tumor and has received a successful treatment, a relapse or recurrence may be the occurrence of a gastric tumor or the occurrence of a tumor at a site different to stomach. A relapse or recurrence of a tumor also includes situations wherein a tumor occurs at a site different to the site of the original tumor as well as at the site of the original tumor. Preferably, the original tumor for which the patient has received a treatment is a primary tumor and the tumor at a site different to the site of the original tumor is a secondary or metastatic tumor.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Examples

### Example 1. Antibody Generation

### 1.1 Immunization and Serum Titer Determination

### 1.1.1 Immunogen and Immunization Strategies

To generate antibodies specifically bind to human CLDN18.2, mice were immunized with a construct expressing full-length human CLDN18.2 protein (pTT5-022-A2, the amino acid sequence of the full-length human CLDN18.2 protein is as set forth in SEQ ID NO: 9) and/or a conjugate comprising a Keyhole limpet hemocyanin (KLH) protein and a peptide fragment of the full-length human CLDN18.2 protein (namely, 996659-KLH, TC-20-KLH, and/or YC-14-KLH). In particular, the amino acid sequence of 996659 peptide fragment is as follows: NNPVTAVFNYQGC (SEQ ID NO: 10); the amino acid sequence of TC-20 peptide fragment is as follows: TQDLYNNPVTAVFNYQGLCC (SEQ ID NO: 11); the amino acid sequence of YC-14 peptide fragment is as follows: YNNPVTAVFNYQGC (SEQ ID NO: 12). “996659-KLH” refers to a 996659 peptide fragment having an amino acid sequence as set forth in SEQ ID NO: 10 conjugated to a KLH protein; by the same token, “TC-20-KLH” refers to a TC-20 peptide fragment having an amino acid sequence as set forth in SEQ ID NO: 11 conjugated to a KLH protein, and “YC-14-KLH” refers to a YC-14 peptide fragment having an amino acid sequence as set forth in SEQ ID NO: 12 conjugated to a KLH protein.

Mice from Shanghai Laboratory Animal Center (SLAC) (female, 6-8 weeks) were fed under Specific Pathogen Free (SPF) conditions. 4 groups of mice (5 mice/group) were immunized by different immunogens. Peripheral blood (PB) sera were collected from all mice before the primary immunization. Except for the first injection, Test blood (TB) serum was collected at 2 or 3 weeks after each injection. To improve immune response, additional boosts were given. Detailed immunization protocols for each group of animals were shown in Tables 3~5 below.

TABLE 3
Grouping of Animals

| | | | Animal/ | Group | |
|---|---|---|---|---|---|
| Group | Immunogen | Route | Strain | size | Adjuvant |
| 1 | 996659-KLH/ | I.P. | Balb/c | 5 | CFA/IFA/IFA/ |
| | TC-20-KLH | | | | IFA |
| 2 | 996659-KLH/ | I.P. | SJL | 5 | CFA/IFA/IFA/ |
| | TC-20-KLH | | | | IFA |
| 3 | pTT5-022-A2/ | Gene | Balb/c | 5 | GM-CSF & |
| | TC-20-KLH/ | gun/ | | | FLT3L/GM-CSF |
| | YC-14-KLH | I.P./I.P. | | | & |
| | | | | | FLT3L/CFA/IFA/ |
| | | | | | IFA |
| 4 | pTT5-022-A2/ | Gene | SJL | 5 | GM-CSF & |
| | TC-20-KLH/ | gun/ | | | FLT3L/GM-CSF |
| | YC-14-KLH | I.P./I.P. | | | & |
| | | | | | FLT3L/CFA/IFA/ |
| | | | | | IFA |

TABLE 4
Immunization protocol for Group 1&2

| | Group 1&2 |
|---|---|
| Day 0 | Pre-bleed (15~30 μL serum/mouse) |
| | Primary immunization |
| | 50 unit/animal |
| | CFA as adjuvant |
| Day 14 | Boost immunization |
| | 25 unit/animal |
| | IFA as adjuvant |
| Day 21 | Test bleed serum collection |
| Day 35 | Boost immunization |
| | 25 unit/animal |
| | IFA as adjuvant |
| Day 42 | Test bleed serum collection |
| Day 63 | Boost immunization |
| | 25 unit/animal |
| | IFA as adjuvant |
| Day 70 | Test bleed serum collection |

TABLE 5
Immunization protocol for Group 3&4

| | Group 3 and 4 |
|---|---|
| Day 0 | Primary immunization |
| | 4 unit/animal |
| | GM-CSF and FLT3L as adjuvant |
| Day 14 | Boost immunization |
| | 4 unit/animal |
| | GM-CSF and FLT3L as adjuvant |
| Day 21 | Test bleed serum collection |
| Day 28 | Boost immunization |
| | 50 unit/animal |
| | CFA as adjuvant |
| Day 35 | Boost immunization |
| | 25 unit/animal |
| | IFA as adjuvant |
| Day 42 | Test bleed serum collection |
| Day 49 | Test bleed serum collection |
| Day 63 | Boost immunization |
| | 25 unit/animal |
| | IFA as adjuvant |
| Day 70 | Test bleed serum collection |

### 1.1.2 Test Bleed Antiserum Analysis

Serum titer was measured by ELISA and FACS. Two proteins, each of which comprises a human CLDN18.2 or human CLDN18.1 peptide fragment and BSA protein (namely, 996656-BSA and 996660-BSA), were used for coating in ELISA-based binding assays. In particular, the 996656 peptide fragment is a human CLDN18.2 peptide fragment, and its amino acid sequence is as follows: TQDLYNNPVTAVFNYQGLWRSC (SEQ ID NO: 13), the 996660 peptide fragment is a human CLDN18.1 peptide fragment, and its amino acid sequence is as follows: STQDLYDNPVTSVFQYEGLWRSC (SEQ ID NO: 14). “996656-BSA” refers to a human CLDN18.2 peptide fragment having an amino acid sequence as set forth in SEQ ID NO: 13 conjugated to a BSA protein; by the same token, “996660-BSA” refers to a human CLDN18.1 peptide fragment having an amino acid sequence as set forth in SEQ ID NO: 14 conjugated to a BSA protein.

The protocol of ELISA assay is described as follows:

Peptides were diluted with coating buffer to 1 μg/mL, and 100 μL of diluted recombinant proteins were coated onto 96-well ELISA plates overnight at 4° C. The plates were washed 3 times with washing buffer (PBST, PBS+0.05% Tween-20, pH 7.4). 300 μL/well blocking buffer (1% BSA in PBST, pH 7.4) was added to each well and incubated for 2 hours at 37° C. Thereafter, 100 μL of antiserum was diluted in blocking buffer, added to each well of the 96-well ELISA plate, and incubated at 37° C. for 1 hour. The plates were washed 3 times with washing buffer. 50 μL/well of anti-mouse IgG Fc-HRP (1:5000 in blocking buffer) were added to each well and incubated for 30 mins at room temperature, and the plates were washed 3 times with washing buffer. 100 μL/well of TMB substrate was added to each well and incubated at room temperature for 15 mins. 50 μL/well of 1 N HCl was added to terminate the reaction, and the absorbance at 450 nm was measured with a microplate reader.

The protocol of FACS binding assay is described as follows:

Stably transfected cell clones CHOK1-hCLDN18.2, CHOK1-hCLDN18.1 and the parental CHOKI cells were digested with TrypLE for 3-5 mins at 37° C. Harvested cells were centrifuged at 400 g for 5 min, and the supernatants were aspirated. Cells were resuspended with 10-15 mL ice-cold culture medium, and cell numbers were counted using a hemocytometer. Cells were centrifuged at 400 g for 5 mins, the supernatant was aspirated. The cells were resuspended in FACS buffer (PBS+2% FBS) at 1.5×10⁶ cells/ml cell. 200 μL of cell suspension were dispensed into each well of the 96-well plate. Cells were centrifuged at 400 g for 5 min, the supernatant was discarded. 200 μL 4% PFA was added and stained at room temperature for 10 mins. Cells were centrifuged at 500 g for 5 mins and the supernatant was discarded. 200 μL 0.2% Triton in PBS was added and incubated at room temperature for 10 mins. Cells were centrifuged at 500 g for 5 mins and the supernatant was discarded. Test bleed sera were diluted to the desired dilution (1:100/1:1000) in FACS buffer. Resuspended cells were incubated with 100 μL/well diluted test bleed serum/tool antibodies (1 μg/mL) for 1 hour at 4° C. Meanwhile, the secondary antibody (Alexa Fluor 488 anti-mouse/rabbit IgG (H+L)/anti-hFc) was diluted in FACS buffer at 1:1000/1:500 dilution. The cells were then washed 3 times with 200 μL PBS and after the final wash, resuspended in 100 μL secondary antibody and incubated for 1 hour at 4° C. in the dark. Cells were washed 2 times with 200 μL PBS by centrifuging at 400 g for 5 mins in a refrigerated centrifuge. The supernatant was discarded, and the cells were resuspended in 100 μL cold PBS and subjected to FACS analysis using a BD FACSCantoII™.

### 1.2 Hybridoma Generation, Screening and Cryopreservation

### 1.2.1 Hybridoma Generation

Myeloma cell preparation: Hybridomas were generated by the fusion of myeloma with splenocyte. Myeloma cells SP2/0 were examined by using an inverted phase microscope to ensure cell viability (>95%) and free of contamination. Pre-fusion cells were counted using a hemocytometer and trypan blue according to the manufacturer's instructions and the results were recorded on Cell Fusion Worksheet. Typically, 2×10⁷ SP2/0 cells were resuspended in 20 mL fusion media.

Splenocyte harvest and culture: Splenocytes were harvested from mice with a strong immune response against 996656-KLH and CHOK1-hCLDN18.2 as determined by serum titer were selected and used for hybridoma generation. Mice selected for fusion and were given a final boost by intraperitoneal injection of TC-20-KLH (25 μg). Three days later, the mice were euthanized by carbon dioxide asphyxiation following an approved IACUC protocol, and a blood sample and splenocytes were collected as per established methods. Serum was generated and used as a positive control (designated as FB) at the hybridoma screening stage. Splenocytes were centrifuged at 400 g for 5 mins and the supernatant was discarded.

Electro-fusion: To isolate splenocytes, spleen was pressed through the screen into fusion medium (DMEM) with a plunger of a sterile disposable syringe. And the cells were resuspended by inverting the centrifuge tube for 3-5 times, centrifuged at 400 g for 5 mins and the supernatant was discarded. Splenocytes were re-suspended in 5 mL Red Blood Cell lysis buffer, incubated for 5 mins at 4° C., the reaction was stopped by the addition of DMEM supplemented with FBS. Splenocytes were then centrifuged at 400 g for 5 mins and re-suspended in DMEM. Following by cells counting, SP2/0 cells (2.5×10⁷ SP2/0 cells per 108 splenocytes) were then added to the splenocytes to achieve a final ratio of splenocytes:SP2/0 of 4:1. The cells were centrifuged at 400 g for 5 mins, and the medium was discarded. The cell pellet was washed twice using electro-fusion (EF) solution and resuspended in the EF solution. The mixture of cells was placed in the fusion slot and the fusion was effectuated by cell fusion generator using an optimized program.

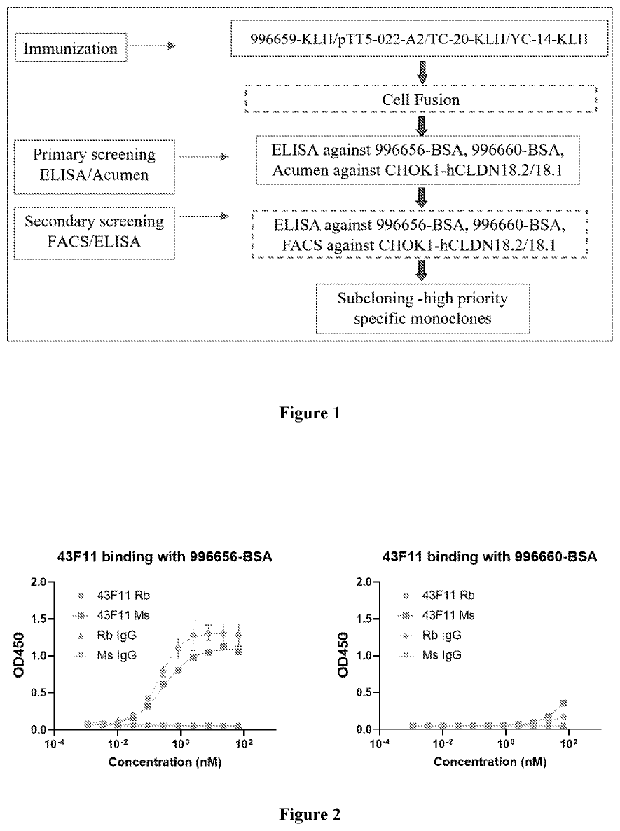

Plating: After electro-fusion, the fused cells were placed in slot for an additional 10 mins. Cells were re-suspended in DMEM with 20% FBS+HT, adjusted to 5×10⁵ cells/mL, and seeded 100 μL/well in a 96-well plate. Plates were carefully transferred to and incubated in a 37° C., 5% CO₂ incubator. 100 μL DMEM containing 20% FBS and 2×HAT was then added at 24 hour post-electro-fusion. Cell growth condition and potential contamination were monitored daily. When cell colonies reached 1-2 mm in diameter (usually 10-14 days post fusion), hybridoma supernatant was collected and screened according to the screening diagram as shown in FIG. 1.

Subcloning and Expansion: Hybridoma clones that exhibited binding to 996656-BSA by ELISA and human CLDN18.2 by Acumen Explorer Microplate Cytometer were expanded into 24-well plates. Hybridoma supernatant was collected from 24-well cultures and the binding ability against 996656-BSA and 996660-BSA peptides was tested by ELISA, and the binding ability to human CLDN18.2 and human CLDN18.1 was evaluated by FACS assay. Clones that specifically bound to 996656-BSA and human CLDN18.2 were selected for subcloning.

Subcloning was performed by limiting dilution for the desired positive parental clones. After incubation at 37° C., 5% CO₂ for 7 days, primary screening assay (by using Acumen Explorer Microplate Cytometer) and secondary screening assay were performed for the subcloned plates. 1-2 rounds of the subcloning process were performed to ensure mono-clonality. Hybridoma monoclonal cells with better binding activity were expanded, frozen, and cryopreserved.

As a result, two fusions were arranged for this project, and the final numbers of hits in the electro-fusion assay were listed in Table 6 below. Briefly, one hit was expanded and cryopreserved among these monoclines.

TABLE 6
Fusion summary

| | | | | | | 1^{st} subclone | | | | |
|---|---|---|---|---|---|---|---|---|---|---|
| | | Primary | | Secondary | | Primary | | Secondary | | |
| | | screening | | screening | | screening | | screening | | |
| Fusion | Mice | ELISA | Acumen | ELISA | FACS | ELISA | Acumen | ELISA | FACS | Frozen |
| F1027A | G1 Balb/c | 30 | 3 | 3 | 3 | 1 | 1 | 1 | 1 | 1 |
| | (12747, 12748) | | | | | | | | | |
| F1027B | G4 SJL | / | / | / | / | / | / | / | / | / |
| | (12761, 12763) | | | | | | | | | |

### 1.2.2 Screening Method

ELISA assay and FACS binding assay were performed as described in Example 1.1.2.

In the primary binding assay done by Acumen Explorer Microplate Cytometer, CHOK1-hCLDN18.2 and CHOK1-hCLDN18.1 (3000 cells/well) were seeded into 384-well plate 1 day before staining. Cells were washed with PBS once and resuspended in 20 μL/well 4% PFA at room temperature and incubated for 10 mins. Cells were washed with PBS once and resuspended in 20 μL/well 0.2% Triton X-100 at room temperature and incubated for 10 mins. Then, cells were washed with PBS once and added 20 μL/well hybridoma supernatants/FB (1:100) solution/primary antibodies and incubated for 2 hours at 37° C. After incubation, cells were washed twice with PBST (PBS+0.05% Tween 20). 20 μl secondary antibody dilution (anti-Mouse IgG Fc specific-FITC antibody produced in goat, 1:500) was added to each well. After incubation for 1 hour at 37° C. and washed twice, cells were stained with 20 μL of propidium iodide (PI, 9 μM) and RNase A (200 μg/mL) solution for 30 mins at 37° C. The signal was detected by Acumen Explorer microplate cytometer.

As a result, several antibodies were evaluated, among which, clone 43F11 and its subclones showed positive binding with fixed CHO-K1 cell stably over-expressing human CLDN18.2 protein (CHOK1-hCLDN18.2), while did not bind with fixed CHO-K1 cell line stably over-expressing human CLDN18.1 protein (CHOK1-hCLDN18.1). In addition, most of 43F11 subclones showed binding ability to human CLDN18.2 peptide (996656-BSA), but did not have binding ability to human CLDN18.1 peptide (996660-BSA). These results suggested that these antibodies specifically recognize human CLDN18.2. The first subclone secondary screening results were summarized in Table 7 below.

TABLE 7
First-subclone secondary screening results

| | Cell line | | | | | |
|---|---|---|---|---|---|---|
| | | | | | 31847 F1027A 1st-subclone | |
| | 31847 F1027A 1st-subclone secondary FACS screening | | | | secondary ELISA screening | |
| | CHOK1-hCLDN18.1 | | CHOK1-hCLDN18.2 | | OD450 nm | |
| | P1 Alexa Fluor | P2% | P1 Alexa Fluor | P2% | 996656-BSA | 996660-BSA |
| Clone NO. | 488 Median | Parent | 488 Median | Parent | (1 μg/ml) | (1 μg/ml) |
| 34A8B11 | 124 | 1.5 | 128 | 1.5 | 0.65 | 0.71 |
| 34A8E11 | 174 | 8.2 | 181 | 7.9 | 1.13 | 1.21 |
| 34A8E5 | 134 | 2.2 | 139 | 2.3 | 0.83 | 0.98 |
| 34A8G11 | 110 | 0.6 | 109 | 0.6 | 0.56 | 0.92 |
| 43F11-2B11 | 364 | 53.5 | 1150 | 97.6 | 1.30 | 0.14 |
| 43F11-2D2 | 485 | 74.5 | 1411 | 98.3 | 1.48 | 0.19 |
| 43F11-2F2 | 588 | 84.6 | 2052 | 99.7 | 1.68 | 0.19 |
| 43F11-2G7 | 400 | 60.9 | 1348 | 98.2 | 1.44 | 0.82 |
| 43F11-3E4 | 521 | 78 | 1636 | 98.3 | 1.66 | 0.17 |
| 43F11-3F11 | 488 | 74.5 | 1115 | 92.1 | 1.56 | 0.16 |
| 43F11-3H3 | 418 | 64.5 | 1328 | 98.7 | 1.43 | 0.20 |
| 43F11-3H6 | 373 | 55.3 | 875 | 95.2 | 1.52 | 0.18 |
| 43F11-4A5 | 431 | 66.3 | 1356 | 98.6 | 1.37 | 0.17 |
| 43F11-4C11 | 527 | 78.6 | 1828 | 99.6 | 1.56 | 0.21 |
| 43F11-4D8 | 638 | 87.6 | 2254 | 99.6 | 1.71 | 0.25 |
| 43F11-4H8 | 553 | 81.6 | 2033 | 99.4 | 1.72 | 0.23 |
| 43F11-5A6 | 426 | 65.8 | 1353 | 98.8 | 1.44 | 0.16 |
| 43F11-5B9 | 492 | 75 | 1625 | 99.2 | 1.51 | 0.50 |
| 43F11-5E4 | 610 | 86.5 | 1802 | 98.9 | 1.69 | 0.65 |
| 43F11-5G10 | 582 | 83.6 | 2404 | 99.6 | 1.70 | 0.20 |
| 43F11C7 | 332 | 46.4 | 1043 | 97 | 1.30 | 0.13 |
| 43F11F10 | 446 | 68.6 | 1416 | 98.9 | 1.43 | 0.15 |
| 43F11F2 | 497 | 76.5 | 1561 | 99 | 1.48 | 0.15 |
| 43F11G5 | 423 | 65.5 | 1426 | 98.3 | 1.46 | 0.25 |
| 9B8C6 | 4020 | 99.8 | 4273 | 99.7 | 0.33 | 0.16 |
| 9B8D9 | 3349 | 99.8 | 3392 | 99.2 | 0.36 | 0.22 |
| 9B8F3 | 3186 | 99.2 | 3015 | 99.5 | 0.45 | 0.22 |
| 9B8G3 | 4052 | 99.8 | 3804 | 99.4 | 0.28 | 0.38 |
| NC | 101 | 0.4 | 102 | 0.5 | 0.11 | 0.21 |
| mIgG | 125 | 1.1 | 118 | 0.7 | 0.10 | 0.22 |
| FB (12747) | 3032 | 99.8 | 2990 | 98.9 | 2.70 | 2.89 |
| FB (12748) | 4244 | 99.7 | 4963 | 98.8 | 2.55 | 2.75 |
| Rabbit IgG | 838 | 97.4 | 892 | 97.5 | 0.39 | 1.31 |
| ab222512 | 397 | 62.9 | 1364 | 98 | 0.16 | 0.00 |

### 1.2.3 Cryopreservation

The desired sub-clonal cell lines were sequenced and further expanded into culture flasks for cryopreservation. Each cell lines stored in 4-6 vials at 0.5-1.0×10⁷ cells/vial were initially cryopreserved. Master cell bank and working cell bank have been established for the selected most valuable cell lines.

### 1.2.4 Sequencing Results

Three subclones (43F11-G5, 43F11-4D8 and 43F11-5B9) were sequenced, and the result showed that they all had the same sequence. The detailed sequence information of antibodies is shown in Table 8 below (the CDR sequences defined by the convention of Kabat and DNA sequences encoding the CDRs are underlined).

TABLE 8
Sequencing results

| Description | Sequences |
|---|---|
| Heavy Chain | DNA Sequence (SEQ ID NO: 17) |
| (FR1 + CDR1 + | GATGTACAGCTGGTGGAGTCTGGGGGAGGTTTAGTGCAG |
| FR2 + CDR2 + | CCTGGAGGGTCCCGGAAACTCTCCTGTGCAGCCTCTGGA |
| FR3 + CDR3 + | TTCACCTTCAGTAGTTATGGAATGCATTGGGTTCGTCAGG |
| FR4) | CTCCAGAGAAGGGGCTGGAGTGGGTCGCATTCATTAGTA |
| | GTGGCAGTAGAACCATCTACTATGCAGACACAGTGAAGG |
| | GCCGATTCACCATCTCCAGAGACAATCCCAAGAATACTC |
| | TGTTCCTGCAAATGACCAGTCTAAGGTCTGAAGACACGG |
| | CCATGTATTACTGTGCAGGATCTACTGGCTGGGACGACTG |
| | GTTTGTTTACTGGGGCCAAGGGACTCTGGTCACTGTCTC |
| | TGCA |
| | Amino Acid Sequence (SEQ ID NO: 7) |
| | DVQLVESGGGLVQPGGSRKLSCAASGFTFSSYGMHWVRQ |
| | APEKGLEWVAFISSGSRTIYYADTVKGRFTISRDNPKNTLFL |
| | QMTSLRSEDTAMYYCAGSTGWDDWFVYWGQGTLVTVSA |
| Light Chain | DNA Sequence (SEQ ID NO: 18) |
| (FR1 + CDR1 + | GATATTGTGATGACTCAGTCTGCACCCTCTGTACCTGTCA |
| FR2 + CDR2 + | CTCCTGGAGAGTCAGTATCCATCTCCTGCAGGTCAAGTA |
| FR3 + CDR3 + | AGAGTCTCCTGCATACTAATGGCAACACTTACTTGTATTG |
| FR4) | GTTCCTGCAGAGGCCAGGCCAGTCTCCTCAAGTCCTGAT |
| | ATATCGGATGTCCAACCTTGCCTCAGGAGTCCCAGACAG |
| | GTTCAGTGGCAGTGGGTCAGGAACTGCTTTCACACTGA |
| | GAATCAGTAGAGTGGAGGCTGACGATGTGGGTGTTTATT |
| | ACTGTATGCAACATCTAGAATATCCTTTAACGTTCGGTGC |
| | TGGGACCAAGCTGGAGCTGAAA |
| | Amino Acid Sequence (SEQ ID NO: 8) |
| | DIVMTQSAPSVPVTPGESVSISCRSSKSLLHTNGNTYLYWF |
| | LQRPGQSPQVLIYRMSNLASGVPDRFSGSGSGTAFTLRISR |
| | VEADDVGVYYCMQHLEYPLTFGAGTKLELK |

### Example 2. Antibody Characterization

### 2.1 Binding Affinity

With the 43F11 sequence, recombinant monoclonal antibodies were engineered with mouse IgG1 (43F11 Ms) or rabbit IgG Fc (43F11 Rb). Binding affinity of the recombinant antibodies to hCLDN18.2-peptide (996656-BSA) and hCLDN18.1-peptide (996660-BSA) were evaluated by ELISA as described in Example 1.1.2. As shown in FIG. 2, both 43F11 Ms and 43F11 Rb had good binding affinity to hCLDN18.2-peptide (IC₅₀ was 0.212 nM and 0.271 nM, respectively), but did not bind to hCLDN18.1-peptide.

### 2.2 Immunohistochemistry

To test the tissue specificity of candidate antibodies, an immunohistochemistry (IHC) assay was performed as follows:
(a) Leica bond III/bond MAX staining machine was used to operate the steps of dewaxing, antigen repair and staining. The antigen retrieval conditions were ER1/ER2 antigen retrieval solution at 100° C. for 20 mins. Samples were washed 3-5 times with TBST after each step between steps (b)-(f).
(b) Endogenous peroxidase activity was blocked by incubating slides in hydrogen peroxide for 10 mins, followed by incubating in blocking solution for 10 mins.
(c) Primary antibody was diluted into different concentrations and incubated at room temperature for 60 mins.
(d) Leica DS9800 staining system secondary antibody was added and incubated at room temperature for 30 mins.
(e) After dipping once with DAB working solution, DAB working solution was added for staining development for another 5 mins.
(f) Hematoxylin working solution was added and stained for 10 mins.
(g) Slides were dehydrated with gradient ethanol and sealed with xylene transparent and neutral gum.
(h) After sealing and imaging by scanner, the IHC results were recorded and analyzed.

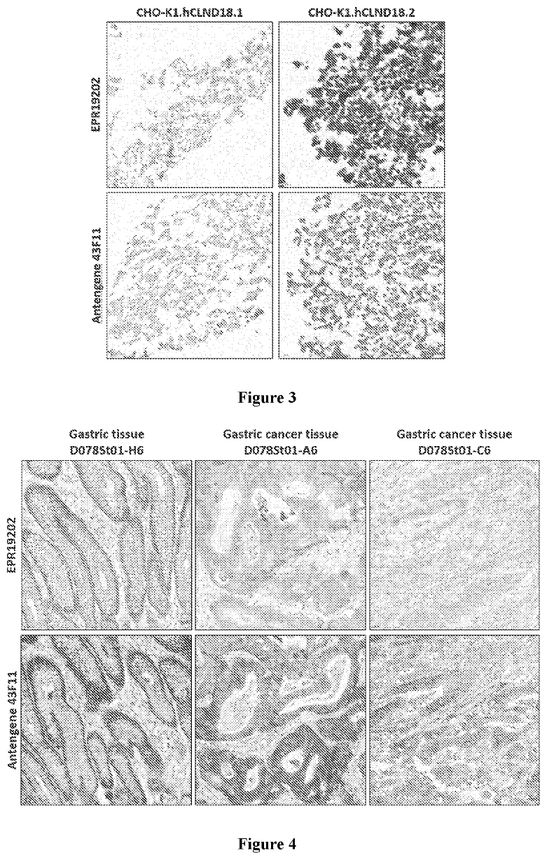

As shown in FIG. 3, antibody 43F11Ms showed positive IHC staining on membrane of CHO-K1.hCLDN18.2 cells (CHO-K1 cells overexpressed human CLDN18.2 protein) after fixation, but showed negative staining on CHO-K1.hCLDN18.1 cells (CHO-K1 cells overexpressed human CLDN18.1 protein). In addition, the IHC staining of CHO-K1.hCLDN18.2 cells by 43F11 antibody showed higher specificity than that of positive control EPR19202 antibody (Abcam, ab222512).

As shown in FIG. 4, in gastric cancer tissue microarray (D079ST01), the antibody 43F11 showed good staining on cell membrane of normal gastric tissue (H6) and gastric cancer tissue (A6, C6), exhibiting higher sensitivity than the positive control EPR19202 antibody. These results suggested that antibody 43F11 could stain tissues with lower CLDN18.2 protein expression levels.

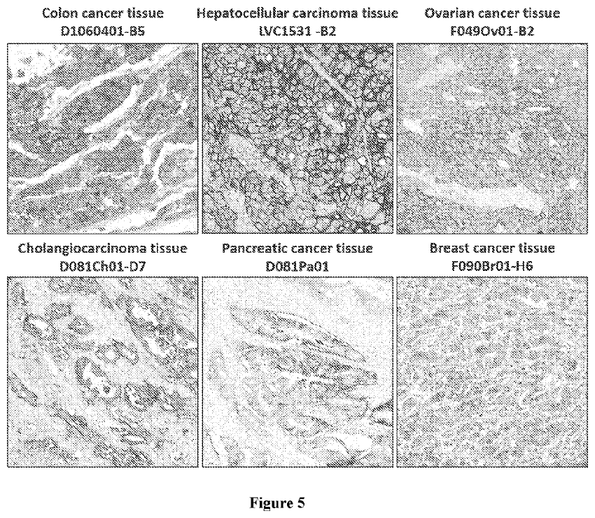

Furthermore, the antibody 43F11 showed excellent positive membrane staining on different cancer tissues expressing CLND18.2 proteins (e.g., colon cancer tissue, hepatocellular carcinoma tissue, ovarian cancer tissue, cholangiocarcinoma tissue, pancreatic cancer tissue, breast cancer tissue), as shown in FIG. 5.

What is claimed is:

1. An antibody or antigen-binding fragment thereof which specifically binds to CLDN18.2, comprising:
one, two or three heavy chain complementarity determining regions (HCDR1, HCDR2 and/or HCDR3) contained within a heavy chain variable (VH) region sequence as set forth in SEQ ID NO: 7; and/or
one, two or three light chain complementarity determining regions (LCDR1, LCDR2 and/or LCDR3) contained within a light chain variable (VL) region sequence as set forth in SEQ ID NO: 8.

2. The antibody or antigen-binding fragment thereof of claim 1, wherein the antibody or antigen-binding fragment comprises at least one heavy or light chain complementarity determining region (CDR) comprising an amino acid sequence selected from the group consisting of SEQ ID NOs: 1, 2, 3, 4, 5 and 6.

3. The antibody or antigen-binding fragment thereof of claim 1 or 2, comprising one, two or three of HCDR1, HCDR2 and HCDR3 comprising an amino acid sequence selected from the group consisting of SEQ ID NOs: 1, 2 and 3.

4. The antibody or antigen-binding fragment thereof of any one of the preceding claims, comprising one, two or three of LCDR1, LCDR2 and LCDR3 comprising an amino acid sequence selected from the group consisting of SEQ ID NOs: 4, 5 and 6.

5. The antibody or antigen-binding fragment thereof of any one of the preceding claims, comprising:
i. a HCDR1 comprising an amino acid sequence as set forth in SEQ ID NO: 1;
ii. a HCDR2 comprising an amino acid sequence as set forth in SEQ ID NO: 2; and
iii. a HCDR3 comprising an amino acid sequence as set forth in SEQ ID NO: 3.

6. The antibody or antigen-binding fragment thereof of any one of the preceding claims, comprising:
i. a LCDR1 comprising an amino acid sequence as set forth in SEQ ID NO: 4;
ii. a LCDR2 comprising an amino acid sequence as set forth in SEQ ID NO: 5; and
iii. a LCDR3 comprising an amino acid sequence as set forth in SEQ ID NO: 6.

7. The antibody or antigen-binding fragment thereof of any one of the preceding claims, comprising:
a HCDR1 comprising an amino acid sequence as set forth in SEQ ID NO: 1,
a HCDR2 comprising an amino acid sequence as set forth in SEQ ID NO: 2,
a HCDR3 comprising an amino acid sequence as set forth in SEQ ID NO: 3,
a LCDR1 comprising an amino acid sequence as set forth in SEQ ID NO: 4,
a LCDR2 comprising an amino acid sequence as set forth in SEQ ID NO: 5,
and a LCDR3 comprising an amino acid sequence as set forth in SEQ ID NO: 6.

8. The antibody or antigen-binding fragment thereof of any one of the preceding claims, comprising a VH region having an amino acid sequence as set forth in SEQ ID NO: 7, or a homologous sequence thereof having at least 80% sequence identity to SEQ ID NO: 7.

9. The antibody or antigen-binding fragment thereof of any one of the preceding claims, comprising a VL region having an amino acid sequence as set forth in SEQ ID NO: 8, or a homologous sequence thereof having at least 80% sequence identity to SEQ ID NO: 8.

10. The antibody or antigen-binding fragment thereof of any one of the preceding claims, comprising a VH region having an amino acid sequence as set forth in SEQ ID NO: 7 and a VL region having an amino acid sequence as set forth in SEQ ID NO: 8.

11. The antibody or antigen-binding fragment thereof of any one of the preceding claims, further comprising one or more amino acid residue substitutions or modifications yet retains specific binding affinity to CLDN18.2.

12. The antibody or antigen-binding fragment thereof of claim 11, wherein at least one of the substitutions or modifications is within one or more of the CDR sequences of the VH region or VL region.

13. The antibody or antigen-binding fragment thereof of claim 11, wherein at least one of the substitutions or modifications is within one or more of the non-CDR sequences of the VH region or VL region.

14. The antibody or antigen-binding fragment thereof of the preceding claims, further comprising one or more non-natural amino acid (NNAA) substitutions.

15. The antibody or antigen-binding fragment thereof of claim 14, wherein the NNAA is capable of being conjugated.

16. The antibody or antigen-binding fragment thereof of any one of the preceding claims, having one or more properties selected from the group consisting of:
a) having substantially no cross-reactivity to human CLDN18.1;
b) having substantially no cross-reactivity to human CLDN18.1 as measured by ELISA, FACS or IHC;
c) having substantially no cross-reactivity to human CLDN18.1 peptide fragment within the amino acid sequence of STQDLYDNPVTSVFQYEGLWRSC (SEQ ID NO: 14) as measured by ELISA;
d) being capable of specifically binding to an epitope within the amino acid sequence of TQDLYNNPVTAVFNYQGLWRSC (SEQ ID NO: 13) as measured by ELISA;
e) being capable of specifically binding to an epitope within the amino acid sequence of human CLDN18.2 expressed in cells as measured by FACS assay; and
f) being capable of specifically binding to human CLDN18.2 expressed in a cancer cell (e.g., a gastric cancer cell, a colon cancer cell, a hepatocellular carcinoma cell, an ovarian cancer cell, a cholangiocarcinoma cell, a pancreatic cancer cell, a breast cancer cell) as measured by IHC.

17. An antibody or antigen-binding fragment thereof, which competes for binding to CLDN18.2 with the antibody or antigen-binding fragment thereof of any one of the preceding claims.

18. The antibody or antigen-binding fragment thereof of any one of the preceding claims, which is a chimeric, a humanized or a human antibody or an antigen-binding fragment thereof.

19. The antibody or an antigen-binding fragment thereof of any one of the preceding claims, which is a monoclonal antibody, a bispecific antibody, a multi-specific antibody, a recombinant antibody, a chimeric antibody, a humanized antibody, a labeled antibody, a bivalent antibody, an anti-idiotypic antibody, a fusion protein, a dimerized or polymerized antibody, or a modified antibody (e.g., glycosylated antibody).

20. The antibody or antigen-binding fragment thereof of any one of the preceding claims, which is a diabody, a Fab, a Fab′, a F(ab′) ₂, a Fd, an Fv fragment, a disulfide stabilized Fv fragment (dsFv), a (dsFv) ₂, a bispecific dsFv (dsFv-dsFv′), a disulfide stabilized diabody (ds diabody), a single-chain antibody molecule (scFv), an scFv dimer (bivalent diabody), a camelized single domain antibody, a nanobody, a domain antibody, or a bivalent domain antibody.

21. The antibody or antigen-binding fragment thereof of any one of the preceding claims, further comprising an Fc region, optionally an Fc region of human immunoglobulin (Ig), or optionally an Fc region of human IgG.

22. The antibody or antigen-binding fragment thereof of claim 21, wherein the Fc region is derived from human IgG1, IgG2, IgG3 or IgG4, mouse IgG1 or rabbit IgG.

23. The antibody or antigen-binding fragment thereof of any one of the preceding claims, wherein the light chain is a λ light chain or a k light chain.

24. The antibody or antigen-binding fragment thereof of claim 22, wherein the Fc region comprises an amino acid sequence of SEQ ID NO: 15, 16, 22, or a sequence having at least 80% sequence identity thereof, and/or the light chain comprises an amino acid sequence of SEQ ID NO: 19, 20, 21 or a sequence having at least 80% sequence identity thereof.

25. The antibody or antigen-binding fragment thereof of any one of the preceding claims, which is linked to one or more conjugate moieties.

26. The antibody or antigen-binding fragment thereof of claim 25, wherein the conjugate moiety comprises a radioactive isotope, a lanthanide, a chemiluminescent label, a chromophoric moiety, colloidal gold particles, a detectable label, a fluorescent label, an enzyme-substrate label, a digoxigenin label, biotin/avidin, a hapten, a DNA molecule for detection, a particle label, a clearance-modifying agent, a chemotherapeutic agent, a toxin, a DNA-alkylator, a topoisomerase inhibitor, a tubulin-binder, a purification moiety or other anticancer drugs.

27. The antibody or antigen-binding fragment thereof of claim 26, wherein the conjugate moiety comprises a biotin or a hapten.

28. The antibody or antigen-binding fragment thereof of claim 26 or 27, wherein the conjugate moiety is covalently attached either directly or via a linker.

29. A chimeric antigen receptor, comprising the antibody or antigen-binding fragment of any one of claims 1-24, a transmembrane region and an intracellular signal region.

30. The chimeric antigen receptor of claim 29, wherein the transmembrane region comprises a transmembrane region of CD3, CD4, CD8 or CD28.

31. The chimeric antigen receptor of claim 29, wherein the intracellular signal region is selected from the group consisting of: an intracellular signal regions sequence of CD3, FcγRI, CD27, CD28, CD137, CD134, MyD88, CD40, CD278, TLRs, or a combination thereof.

32. The chimeric antigen receptor of any one of claims 29-31, wherein the antigen-binding fragment is a scFv.

33. The chimeric antigen receptor of any one of claims 29-32, wherein the chimeric antigen receptor is grafted onto an allogeneic cell, an autologous cell or a xenogeneic cell.

34. The chimeric antigen receptor of any one of claims 29-33, wherein the chimeric antigen receptor is grafted onto an immune effector cell.

35. The chimeric antigen receptor of any one of claims 29-34, wherein the chimeric antigen receptor is grafted onto a T cell, a natural killer cell, a macrophage cell, or a tumor-infiltrating lymphocyte.

36. A pharmaceutical composition comprising the antibody or an antigen-binding fragment thereof of any one of claims 1-28, or the chimeric antigen receptor of any one of claims 29-35, and one or more pharmaceutically acceptable carriers.

37. An isolated polynucleotide encoding the antibody or an antigen-binding fragment thereof of any one of claims 1-28, and/or the chimeric antigen receptor of any one of claims 29-35.

38. A vector comprising the isolated polynucleotide of claim 37.

39. A host expression system comprising the vector of claim 38, or having the polynucleotide of claim 37 integrated into genome thereof.

40. The host expression system of claim 39, which is a microorganism, a yeast, or a mammalian cell, optionally, wherein the microorganism is selected from the group consisting of E. coli and B. subtilis, optionally wherein the yeast is Saccharomyces, and optionally wherein the mammalian cell is selected from the group consisting of COS, CHO—S, CHO-K1, HEK-293, and 3T3 cells.

41. A kit comprising the antibody or antigen-binding fragment thereof of any one of claims 1-28, and/or the chimeric antigen receptor of any one of claims 29-35, and/or the pharmaceutical composition of claim 36.

42. The kit of claim 41, further comprising a second agent.

43. The kit of claim 42, wherein the second agent is a diagnostic agent.

44. The kit of claim 41, further comprising a set of reagents for detecting a complex of the antibody or antigen-binding fragment thereof bound to CLDN18.2.

45. The kit of claim 44, wherein the set of reagents comprises a secondary antibody that binds to the antibody or antigen-binding fragment thereof, optionally the secondary antibody is detectably labeled.

46. The kit of claim 44, wherein the set of reagents comprises a detectably labeled avidin or steptavidin.

47. The kit of claim 41, wherein the antibody or antigen binding fragment thereof is conjugated with an indirectly detectable moiety.

48. The kit of claim 47, wherein the indirectly detectable moiety comprises biotin.

49. A method of expressing the antibody or antigen-binding fragment thereof of any one of claims 1-28 or the chimeric antigen receptor of any one of claims 29-35, comprising culturing the host expression system of claim 39 under the condition at which the antibody or antigen-binding fragment of any one of claims 1-28 or the chimeric antigen receptor of any one of claims 29-35 is expressed.

50. A method of detecting presence or expression level of CLDN18.2 in a sample, comprising contacting the sample with the antibody or antigen-binding fragment thereof of any of claims 1-28 under a condition that allows specific binding of the antibody or antigen-binding fragment thereof to CLDN18.2, and determining presence or expression level of CLDN18.2 in the sample.

51. A method for diagnosing a CLDN18.2-associated disease, disorder or condition (e.g., cancer), screening for the onset or risk to the onset of a CLDN18.2-associated disease, disorder or condition (e.g., cancer), or assessing the development or prognosis of a CLDN18.2-associated disease, disorder or condition (e.g., cancer) in a subject, comprising:
a) contacting a sample obtained from the subject with the antibody or antigen-binding fragment thereof of any of claims 1-28 or the chimeric antigen receptor of any one of claims 29-35 under a condition that allows specific binding of the antibody or antigen-binding fragment thereof or the chimeric antigen receptor to CLDN18.2; and
b) determining presence or expression level of CLDN18.2 in the sample;
wherein the subject is diagnosed as having a CLDN18.2-associated disease, disorder or condition (e.g., cancer), or is at the onset or at a risk to the onset of a CLDN18.2-associated disease, disorder or condition (e.g., cancer), or develops or with an increased probability of developing a CLDN18.2-associated disease, disorder or condition (e.g., cancer), or has poor prognosis or at a risk to poor prognosis of a CLDN18.2-associated disease, disorder or condition (e.g., cancer) when the presence of CLDN18.2 is found or when the expression level of CLDN18.2 equals to or is higher than a threshold level.

52. A method for determining the eligibility of a subject having or at risk of having a CLDN18.2-associated disease, disorder or condition for treatment with a CLDN18.2-targeting agent, comprising:
a) contacting a sample obtained from the subject with the antibody or antigen-binding fragment thereof of any of claims 1-28 or the chimeric antigen receptor of any one of claims 29-35 under a condition that allows specific binding of the antibody or antigen-binding fragment thereof or the chimeric antigen receptor to CLDN18.2; and
b) determining presence or expression level of CLDN18.2 in the sample;
wherein the subject is determined as eligible for treatment with a CLDN18.2-targeting agent when the presence of CLDN18.2 is found or when the expression level of CLDN18.2 equals to or is higher than a threshold level, or
wherein the subject is determined as not eligible for treatment with a CLDN18.2-targeting agent when the presence of CLDN18.2 is not found or when the expression level of CLDN18.2 is lower than a threshold level.

53. A method of predicting therapeutic effectiveness of a CLDN18.2-targeting agent in treating a CLDN18.2-associated disease, disorder or condition in a subject, comprising:
a) contacting a sample obtained from the subject with the antibody or antigen-binding fragment thereof of any of claims 1-28 or the chimeric antigen receptor of any one of claims 29-35 under a condition that allows specific binding of the antibody or antigen-binding fragment thereof or the chimeric antigen receptor to CLDN18.2;
b) determining presence or expression level of CLDN18.2 in the sample; and
c) predicting the therapeutic effectiveness of the CLDN18.2-targeting agent,
wherein the CLDN18.2-targeting agent is predicted to be effective in treating the subject when the presence of CLDN18.2 is found or when the expression level of CLDN18.2 equals to or is higher than a threshold level, or
wherein the CLDN18.2-targeting agent is predicted to be not effective in treating the subject when the presence of CLDN18.2 is not found or when the expression level of CLDN18.2 is lower than the threshold level.

54. A method of treating, preventing or alleviating a CLDN18.2-associated disease, disorder or condition in a subject having or at risk of having the CLDN18.2-associated disease, disorder or condition, comprising:
a) selecting a subject that is suitable for the treatment, prevention or alleviation, comprising:
(i) contacting a sample obtained from the subject with the antibody or antigen-binding fragment thereof of any of claims 1-28 or the chimeric antigen receptor of any one of claims 29-35 under a condition that allows specific binding of the antibody or antigen-binding fragment thereof or the chimeric antigen receptor to CLDN18.2;
(ii) determining the presence or expression level of CLDN18.2 in the sample; and
(iii) selecting the subject as suitable for the treatment, prevention or alleviation of the CLDN18.2-associated disease, disorder or condition when the presence of CLDN18.2 is found or when the expression level of CLDN18.2 in the sample equals to or is higher than a threshold level;
b) administering a therapeutically effective amount of CLDN18.2-targeting agent to the selected subject.

55. A method of treating, preventing or alleviating a CLDN18.2-associated disease, disorder or condition in a subject having or at risk of having the CLDN18.2-associated disease, disorder or condition, comprising:
a) selecting a subject that is suitable for the treatment, prevention or alleviation, comprising:
(i) contacting a sample obtained from the subject with the antibody or antigen-binding fragment thereof of any of claims 1-28 or the chimeric antigen receptor of any one of claims 29-35 under a condition that allows specific binding of the antibody or antigen-binding fragment thereof or the chimeric antigen receptor to CLDN18.2;
(ii) determining the presence or expression level of CLDN18.2 in the sample; and
(iii) selecting the subject as not suitable for the treatment, prevention or alleviation of the CLDN18.2-associated disease, disorder or condition with a CLDN18.2-targeting agent when the presence of CLDN18.2 is not found or when the expression level of CLDN18.2 in the sample is lower than a threshold level;
b) administering to the selected subject a Standard-of-Care therapeutic other than a CLDN18.2-targeting agent.

56. The method of any one of claims 50-55, wherein the sample is selected from the group consisting of a cell sample, a tissue sample, biopsy, a paraffin-embedded tissue, a body fluid (e.g., whole blood, blood serum, blood plasma, urine, mucus, saliva, peritoneal fluid, pleural fluid, chest fluid, synovial fluid, cerebrospinal fluid, thoracentesis fluid, abdominal fluid, and any combination thereof), colonic effluent, a surgical resection sample, an isolated blood cell, a cell isolated from blood, and any combination thereof.

57. The method of claim 56, wherein the sample is a fixed tissue sample, optionally a formalin-fixed paraffin-embedded (FFPE) tissue sample.

58. The method of any one of claims 50-57, wherein the CLDN18.2 is cell surface or membrane-bound CLDN18.2.

59. The method of any one of claims 50-58, wherein the presence or expression level of CLDN18.2 is determined by immunohistochemistry (IHC), Immunocytochemistry (ICC), immunofluorescence (IF), enzyme immunoassay (EIA), Enzyme-Linked Immunosorbent Assay (ELISA), immunochromatography, or immunoblotting.

60. The method of any one of claims 50-59, wherein the expression level of CLDN18.2 is quantified based on percentage of positively stained cells in the sample.

61. The method of any one of claims 50-59, wherein the expression level of CLDN18.2 is quantified based on staining intensity for CLDN18.2 in the sample.

62. The method of any one of claims 51-61, wherein the CLDN18.2-associated disease, disorder or condition is cancer.

63. The method of claim 62, wherein the sample comprises a tumor sample.

64. The method of claim 63, wherein the tumor sample comprises a tumor tissue or a circulating tumor cell.

65. The method of claim 64, wherein the cancer is primary cancer or metastatic cancer.

66. The method of any one of claims 62-65, wherein the cancer is gastric cancer, colon cancer, hepatocellular carcinoma, ovarian cancer, cholangiocarcinoma, pancreatic cancer, breast cancer, lung cancer (e.g., non-small cell lung cancer or small cell lung cancer), bronchial cancer, bone cancer, liver and bile duct cancer, liver cancer, testicle cancer, kidney cancer, bladder cancer, head and neck cancer, spine cancer, brain cancer, cervix cancer, uterine cancer, endometrial cancer, colorectal cancer, rectal cancer, anal cancer, esophageal cancer, gastrointestinal cancer, skin cancer, prostate cancer, pituitary cancer, vagina cancer, thyroid cancer, glioblastoma, astrocytoma, melanoma, myelodysplastic syndrome, sarcoma, teratoma, or adenocarcinoma.

67. The method of claim 66, wherein the cancer is gastric cancer, colon cancer, hepatocellular carcinoma, ovarian cancer, cholangiocarcinoma, pancreatic cancer, breast cancer, or lung cancer (e.g., non-small cell lung cancer or small cell lung cancer).

68. The method of any of claims 52-67, wherein the CLDN18.2-targeting agent is capable of inducing cytotoxicity to CLDN18.2-expressing cells.

69. The method of any of claims 52-68, wherein the CLDN18.2-targeting agent is a therapeutic anti-CLDN18.2 antibody or an CLDN18.2-binding molecule (e.g., anti-CLDN18.2 antibody conjugated to a cytotoxic agent, or a bispecific antibody), a CLDN18.2-targeting cell therapy (e.g., a CAR-T, TCR-T or CAR-NK cell expressing a CLDN18.2-binding CAR), a chemical compound targeting CLDN18.2, or a therapeutic nucleic acid targeting CLDN18.2.

70. The method of any one of claims 51-69, wherein the subject is receiving or has received anti-cancer therapy, or suffers from cancer recurrence.

71. The method of any one of claims 50-70, wherein the CLDN18.2 is human CLDN18.2.

72. The method of any one of claims 51 to 71, wherein the subject is human.


SEQUENCE LISTING (23 sequences)

SEQ ID NO: 1 - protein, 5 aa, synthetic construct
SYGMH

SEQ ID NO: 2 - protein, 17 aa, synthetic construct
FISSGSRTIYYADTVKG

SEQ ID NO: 3 - protein, 10 aa, synthetic construct
STGWDDWFVY

SEQ ID NO: 4 - protein, 16 aa, synthetic construct
RSSKSLLHTNGNTYLY

SEQ ID NO: 5 - protein, 7 aa, synthetic construct
RMSNLAS

SEQ ID NO: 6 - protein, 9 aa, synthetic construct
MQHLEYPLT

SEQ ID NO: 7 - protein, 119 aa, synthetic construct
DVQLVESGGGLVQPGGSRKLSCAASGFTFSSYGMHWVRQAPEKGLEWVAFISSGSRTIYYADTVKGRFTISRDNPKNTLFLQMTSLRSEDTAMYYCAGSTGWDDWFVYWGQGTLVTVSA

SEQ ID NO: 8 - protein, 112 aa, synthetic construct
DIVMTQSAPSVPVTPGESVSISCRSSKSLLHTNGNTYLYWFLQRPGQSPQVLIYRMSNLASGVPDRFSGSGSGTAFTLRISRVEADDVGVYYCMQHLEYPLTFGAGTKLELK

SEQ ID NO: 9 - protein, 261 aa, synthetic construct
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 10 - protein, 13 aa, synthetic construct
NNPVTAVFNYQGC

SEQ ID NO: 11 - protein, 20 aa, synthetic construct
TQDLYNNPVTAVFNYQGLCC

SEQ ID NO: 12 - protein, 14 aa, synthetic construct
YNNPVTAVFNYQGC

SEQ ID NO: 13 - protein, 22 aa, synthetic construct
TQDLYNNPVTAVFNYQGLWRSC

SEQ ID NO: 14 - protein, 23 aa, synthetic construct
STQDLYDNPVTSVFQYEGLWRSC

SEQ ID NO: 15 - protein, 214 aa, synthetic construct
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 16 - protein, 217 aa, synthetic construct
VPELLGGPSVFIFPPKPKDTLMISRTPEVTCVVVDVSQDDPEVQFTWYINNEQVRTARPPLREQQFNSTIRVVSTLPITHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 17 - DNA, 357 bp, synthetic construct
  source 1..357: mol_type = other DNA
gatgtacagctggtggagtctgggggaggtttagtgcagcctggagggtcccggaaactctcctgtgcagcctctggattcaccttcagtagttatggaatgcattgggttcgtcaggctccagagaaggggctggagtgggtcgcattcattagtagtggcagtagaaccatctactatgcagacacagtgaagggccgattcaccatctccagagacaatcccaagaatactctgttcctgcaaatgaccagtctaaggtctgaagacacggccatgtattactgtgcaggatctactggctgggacgactggtttgtttactggggccaagggactctggtcactgtctctgca

SEQ ID NO: 18 - DNA, 336 bp, synthetic construct
  source 1..336: mol_type = other DNA
gatattgtgatgactcagtctgcaccctctgtacctgtcactcctggagagtcagtatccatctcctgcaggtcaagtaagagtctcctgcatactaatggcaacacttacttgtattggttcctgcagaggccaggccagtctcctcaagtcctgatatatcggatgtccaaccttgcctcaggagtcccagacaggttcagtggcagtgggtcaggaactgctttcacactgagaatcagtagagtggaggctgacgatgtgggtgtttattactgtatgcaacatctagaatatcctttaacgttcggtgctgggaccaagctggagctgaaa

SEQ ID NO: 19 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 107 aa, synthetic construct; identical to SEQ ID NO: 19

SEQ ID NO: 21 - protein, 107 aa, synthetic construct [sequence omitted]

SEQ ID NO: 22 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 261 aa, synthetic construct
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV